(19) United States
(12) Patent Application Publication
(10) Pub. No.: US 2014/0105867 A1
(43) Pub. Date: Apr. 17, 2014

(54) BLOCKAGE OF PAI-1 IN DIABETIC CD34+ STEM CELLS CORRECTS CELLULAR DYSFUNCTION

(71) Applicants: Maria Grant, Archer, FL (US); Stephen Bartelmez, Sausalito, CA (US)

(72) Inventors: Maria Grant, Archer, FL (US); Stephen Bartelmez, Sausalito, CA (US)

(73) Assignee: UNIVERSITY OF FLORIDA RESEARCH FOUNDATION. INC., Gainesvile, FL (US)

(21) Appl. No.: 14/006,770

(22) PCT Filed: Mar. 23, 2012
(86) PCT No.: PCT/US12/30387

§ 371 Date: Jan. 3, 2014

(30) Foreign Application Priority Data
Mar. 23, 2011 (US) 61466756

(51) Int. Cl. A61K 35/14

(52) U.S. Cl. CPC A61K 35/14
USPC 424/93.7

(57) ABSTRACT

Disclosed herein are methods of enhancing repair of vascular lesions involving the administration of cells in which PAI-1 expression and/or activity has been transiently blocked. Other methods involve the administration of a PAI-1 blocking agent to a subject who has a vascular lesion or is at risk of developing a vascular lesion. Alternatively, a PAI-1 blocking agent and treated cells are co-administered to a subject in need thereof.

### GOVERNMENT SUPPORT

This work was supported by NIH grants 1RC1EY020341-01, 1R43EY020030-01, 1R43HL093955-01A1, EY007739, EY012601, U01 HL087366, and DK090730, 1R43HL093955, DK48708. The government has certain rights in this invention.

### BACKGROUND

Circulating bone marrow (BM)-derived cells have been shown to play an important role in normal physiologic maintenance and repair of the body's vasculature with approximately 1-12% of endothelial cells at any one time being BM-derived (Schatteman, G. C. Adult bone marrow-derived hemangioblasts, endothelial cell progenitors, and EPCs, Curr Top Dev Biol 64, 141-80 (2004)). BM derived cells can differentiate into endothelial cells, and these cells are thought to be important in processes such as vasculogenesis and vascular repair.

The ability to repair vascular damage could have a profound impact on diabetes-induced complications. Diabetes affects 20 million Americans or about 7 percent of the population. Diabetic complications include heart disease, stroke, kidney failure, blindness, as well as nerve and peripheral vascular disease that can lead to lower limb amputations. Furthermore, preventing diabetic complications could save $2.5 billion annually.

Recent evidence suggests that hematopoietic stem cells (HSC) differentiate into vascular structures as well as into all hematopoietic cell lineages and this has spawned the era of cellular therapies for vascular insufficiency. These therapies are now attempting to replace traditional approaches such as stents, angioplasty, and vessel grafts currently being used to alleviate tissue ischemia (Losordo, D. W. & Dimmeler, S. Therapeutic angiogenesis and vasculogenesis for ischemic disease: part II: cell-based therapies, Circulation 109, 2692-7 (2004)). In diabetic subjects, the entire diabetic endothelium suffers damage as a result of oxidative stress and hyperglycemia. Dysfunction of human diabetic CD34+ endothelial progenitor cells limits autologous cell therapy for vascular complications. Injured macrovasculature endothelium, if not repaired, leads to a propensity for arteriosclerosis. With regard to the microvasculature, this same endothelial damage results in capillary damage in the heart, nerves, skin, and retina (Kugler, C. F. & Rudofsky, G. The challenges of treating peripheral arterial disease, Vasc Med 8, 109-14 (2003)).

In capillaries, a defect in endothelial progenitor cells (EPCs) could prevent reparation of endothelial injury early on, leading to tissue ischemia. In the macrovasculature this same inability to repair the endothelium results in an increase in cytokines and up regulation of adhesion molecules with an influx of lipoprotein, monocytes, and T cells, initiating the atherosclerotic lesion (Ross, R., Glomset, J. & Harker, L. Response to injury and atherogenesis, Am J Pathol 86, 675-84 (1977)). Thus, the cause of diabetic microvascular and macrovascular dysfunction may be the same: a lack of EPC repair of the endothelium. Tissue ischemia may be either retinal or sub-retinal ischemia in many cases, which contribute to visual impairment and blindness in diseases as diverse as retinopathy of prematurity, diabetic retinopathy and age-related macular degeneration.

Vascular disease is a major cause of morbidity and mortality in diabetics worldwide. CD34+ endothelial progenitor cells are biomarkers that predict cardiovascular disease and the metabolic syndrome [1, 2]. Reduction of circulating CD34+ cells predicts the clinical onset of type 2 diabetes [3]. Altered in vitro and in vivo function of progenitor cells is characteristic of patients with diabetic complications [4-9]. While control of CD34+ cell fate is complex, TGF-β is a primary regulator of long-term repopulating-hematopoietic stem cell (LTR-HSC) quiescence (G0) in bone marrow niches [10]. HSC/progenitor cells can be released from G0 by exposure to TGF-β neutralizing antibodies that, in turn, provides for improved retroviral gene transfer [11, 12]. Inhibition of TGF-β signaling downstream of the activated receptor by blocking Smad effector function promotes HSC self-renewal in vivo [13]. TGF-β1 directly and reversibly inhibits growth of murine long-term repopulating HSC (LTR-HSC) and of hematopoietic progenitor cells in vitro [14, 15]. Low numbers of LTR-HSC exposed to neutralizing anti-TGF-β antibodies just prior to transplant greatly enhances the bone marrow rescue of mice after lethal irradiation. Recently, we reported that transient downregulation and functional inhibition of the intracellular TGF-β1 pathway in diabetic human CD34+ cells corrects key dysfunctional behaviors [16] likely through effects on critical TGF-β1 target genes. Recent data, in fact, confirm the role of one such TGF-β1-regulated gene, plasminogen activator inhibitor-1 (PAI-1; SERPINE1) as an important regulator of cellular growth arrest [17]. Levels of PAI-1, a major gene product of TGF-β1 activation, are increased in diabetes, atherosclerosis and obesity [18]. PAI-1 expression is influenced by specific cytokines and growth factors and its activity is regulated at the transcriptional level [19]. PAI-1 expression, like TGF-β negatively regulates PI3K/Akt mediating cell survival, proliferation and migration [20-22]. Furthermore, the absence of PAI-1 protects diabetic animals from development of microvascular complications [23].

### SUMMARY

Transforming growth factor-beta 1 (TGF-β1) is a pleiotropic regulator of all stages of hematopoiesis (Ruscetti, F. W. & Bartelmez, S. H. Transforming growth factor beta, pleiotropic regulator of hematopoietic stem cells: potential physiological and clinical relevance, Int J Hematol 74, 18-25 (2001)). HSC express and secrete active forms of TGF-β (Ruscetti, F. W., Akel, S. & Bartelmez, S. H. Autocrine transforming growth factor-beta regulation of hematopoiesis: many outcomes that depend on the context. Oncogene 24, 5751-63 (2005)). The three mammalian isoforms (TGF-β1, 2 and 3) have distinct but overlapping effects on hematopoiesis, but TGF-β1 is the predominately expressed gene in HSC. Depending on the differentiation stage of the target cell, the local environment, and the concentration of TGF-β, in vivo or in vitro, TGF-β can be pro- or anti-proliferative, pro- or anti-apoptotic, induce or inhibit differentiation, and can inhibit or increase terminally differentiated cell function. Plasminogen activator inhibitor-1 (PAI-1) is a major gene product of TGF-β1. Expression of PAI-1 is increased in endothelial cells by high glucose and insulin exposure, and PAI-1 is increased in serum of diabetics. Described herein for the first time is the inventors' discovery that the beneficial effects of transient inhibition of TGF-β1 on CD34⁺ cell function is mediated by PAI-1 inhibition, and that in some embodiments disclosed herein, blocking PAI-1 corrects diabetes-associated cellular dysfunction.

It was determined herein that the TGF-β/PAI-1 system plays a critical role in HSC/CD34+ cell function and, therefore, effective regulation of this system in the context of diabetes might confer protection from vascular complications. The inventors examined a unique cohort of diabetic patients that had a lifetime of poor glycemic control but remained free of vascular complications to gain insight into the physiological function of TGF-β-PAI-1 network.

The inventors also examined the CD34+ endothelial progenitors (EPCs) from diabetic patients free of microvascular complications despite longstanding poorly control diabetes. It was determined herein that these patients had a unique progenitor population that was able to maintain vascular repair in the presence of chronic endothelial injury. Using gene array studies, it was found that diabetics, protected from vascular complications had reduced level of TGF-β1 and PAI-1 transcripts in their circulating CD34+ cells. Treatment with neutralizing antibody to TGF-β1 in murine HSC enhanced in vivo repopulation potential of HSCs in bone marrow transplantation; reduced the time required for cell division of single cells, increased survival of the progenitor cells and reduced TGF-β1 expression. TGF-β1 phosphorodiamidate morpholino oligomers (PMO) treatment reduced PAI-1 mRNA expression in diabetic (p<0.01) and non-diabetic (p=0.05) CD34+ cells. PAI-1 was inhibited in these cells using lentivirus expressing PAI-1 shRNA, PAI-1 siRNA, or over-expression of miR-146a. Inhibition of PAI-1 promoted CD34+ cell proliferation, migration in vitro and bypassed inhibitory effects of exogenous TGF-β1 on cell survival (p<0.001) even in the absence of growth factors. Targeting the TGF-β1/PAI-1 system provides a therapeutic strategy for restoring vascular reparative function in diabetic progenitor cells, never heretofore identified as such, making autologous cell therapy feasible in diabetic individuals.

In one embodiment, there is provided a method of treating vascular lesions in a subject in need thereof. The method including procuring hematopoietic stem cells from the subject to obtain procured hematopoietic stem cells, treating the procured hematopoietic stem cells, ex vivo, by blocking activity of PAI-1 in the stem cells to obtain treated hematopoietic stem cells, and administering the treated hematopoietic stem cells to the subject.

In another embodiment, there is provided a method of diminishing diabetic retinopathy in a subject including administering hematopoietic stem cells treated with a PAI-1 blocking agent to the subject.

In yet another embodiment, a method of enhancing repair of vessel lesion in a subject including administering hematopoietic stem cells treated with a PAI-1 blocking agent to the subject is provided.

In still another embodiment, a method of treating a condition in a patient in need thereof is provided. The method includes administering to the patient a therapeutically effective amount of a PAI-1 blocking agent, and, optionally, co-administering stem cells subjected, ex vivo, to a PAI-1 blocking agent, wherein the condition is a vessel lesion.

In another embodiment, a method of treating vascular lesions in a subject in need thereof is provided. The method includes procuring umbilical cord blood hematopoietic stem cells, treating the procured hematopoietic stem cells, ex vivo, by blocking activity of PAI-1 in the cells to obtain treated hematopoietic stem cells, and administering the treated hematopoietic stem cells to the subject.

In still another embodiment, a method of treating diabetic ulcers by administering treated hematopoietic stem cells (HSCs) to a patient experiencing a diabetes related wound is provided. The method includes administering an effective amount of treated HSCs to the patient in a manner to deliver the treated HSCs to the wound or vicinity of the wound.

In yet another embodiment, a lesion treating composition including treated hematopoietic stem cells is provided. The treated hematopoietic stem cells are obtained by procuring hematopoietic stem cells from a subject and treating the procured hematopoietic stem cells, ex vivo, by blocking activity of PAI-1 in the cells to obtain treated hematopoietic stem cells.

### BRIEF DESCRIPTION OF THE DRAWINGS

FIG. 1A-D provides a graphical illustration of correlations between PAI-1 and TGF-β1 concentrations and Type 1 and Type 2 diabetes.

FIG. 2A provides a pathway map showing alteration in gene expressions in patients protected from development of microvascular complications compared to the diabetic patients who developed microvascular complications.

FIG. 2B shows a bar chart demonstrating log fold change for respective gene. In this patient population TGF-β1, TGF-βR1, TGF-βR2, PAI-1 and tPA (tissue plasminogen activator) are down-regulated whereas uPA (urokinase plasmonigen activator) is up-regulated.

FIG. 3 shows an increased secreted level of PAI-1 from type 2 diabetic CD34⁺ cells. The CD34⁺ cells from both diabetic and non-diabetic individuals were characterized in terms of their ability to release PAI-1 in the conditioned media (CM); the diabetic cells showed a significantly higher level of secreted PAI-1 compared to non-diabetic cells (p<0.05).

FIG. 4A shows both diabetic and non-diabetic CD34⁺ cells treated with TGF-β1 PMO demonstrated decreased PAI-1 mRNA expression compared to cells treated with scrambled PMO (n=10 for diabetic and n=3 for non-diabetic, p<0.001 for diabetic and p=0.05).

FIG. 4B shows TGF-β1 mediation of the inhibitory effects on CD34⁺ cells survival by PAI-1.

FIG. 5A shows that non-diabetic CD34⁺ cells proliferated following PAI-1 blockade to a greater degree than cells treated with lenti shRNA control.

FIG. 5B shows that diabetic CD34⁺ cells did not proliferate even in the presence of growth factors; however, PAI-1 blockade increased the proliferation rate of the diabetic cells to the level of non-diabetic cells.

FIG. 5C demonstrates that in comparison to control siRNA treated cells, the inhibition of PAI-1 allowed a greater number of cells to survive in the absence of growth factors for 6 days.

FIG. 6A provides that cells infected with PAI-1 siRNA demonstrated greater migratory response compared to the cells treated with scrambled siRNA, suggesting that reducing PAI-1 in the diabetic cells improved their migratory ability in vitro.

FIG. 6B demonstrates that blocking PAI-1 stimulated PI3K activity significantly compared to the activity when the cells were treated with scrambled siRNA (p<0.05).

FIG. 7A shows a successful transfection after healthy CD34⁺ cells were transfected with 20 nM, 40 nM and 60 nM miR-146a mimic for 24 hrs, as a significant increase in miR-146a expression in miR-146a transfected cells than the untreated cells is provided.

FIG. 7B shows cells transfected with miR-146a mimic produced ˜7 times less PAI-1 transcripts in comparison to untreated cells 24 hrs after transfection, suggesting that up regulation of miR-146a can reduce the PAI-1 mRNA expression in CD34⁺ cells.

FIG. 7C provides that over expressing miR-146a also reduced secreted level of PAI-1 in the CM of non-diabetics.

FIG. 7D shows that over expressing miR-146a also reduced secreted level of PAI-1 in the CM of diabetics, although, for diabetics the concentration of miR-146a mimic used was higher than for non diabetics, as the basal level of PAI-1 was higher in diabetics.

FIG. 8A provides a graphical representation of aurvival of single LTR-HSC in serum-free media in the absence of added cytokines and presence of anti-TGF-β1,2 and control and serum free or serum containing.

FIG. 8B is a graphical representation of anti-anti-TGF-β1,2 (1D11.16) reduces expression levels of GFP driven by a TGF-β promoter construct (Ad5-TGF-b-GFP) transduced into lin−c-kit+/Sca-1+ cells.

FIG. 9 provides a graphical illustration of LTR-HSC(CD45.2) treated for 2 hrs with anti-TGF-β1, 2 at 0 weeks-9 months.

FIG. 10A provides a graphical representation of PAI-1 concentration in the conditioned media of diabetic and non-diabetic CD34⁺ cells.

FIG. 10B provides a graphical illustration of the percent decrease of PAI-1 mRNA compared to control in both non-diabetic and diabetic cells.

FIGS. 10 C-D provides a graphical illustration of plasma concentrations of PAI-1 and TGF-β1 in type 1 and type 2 diabetics.

FIG. 11A provides a graphical illustration where non-diabetic CD34+ cells were infected with lentivirus expressing PAI-1 shRNA or scrambled shRNA.

FIG. 11B provides a graph showing Non-diabetic CD34+ cells were infected with either lentivirus expressing PAI-1 shRNA (solid line) or lentivirus expressing scrambled shRNA.

FIG. 11C provides a graph representing diabetic CD34+ cells which were infected with either lentivirus expressing PAI-1 shRNA (solid line) or lentivirus expressing scrambled shRNA.

FIG. 12A provides a graphical representation PI3 kinase activity was measured in nondiabetic CD34+ cells by measuring the amount of PI (3,4,5) P3 produced from PI(4,5) P2 following PAI-1 inhibition.

FIG. 12B provides a graphical illustration of cGMP production after PAI-1 inhibition was measured by chemiluminescence assay.

FIG. 12C provides a graphical representation Boyden chamber assay showing migration of diabetic CD34+ cells to 100 nM of SDF-1α.

### DETAILED DESCRIPTION

As aforementioned, endothelial progenitor cells play a major role in angiogenesis (Asahara, Takayuki., et al., Isolation of Putative Progenitor Endothelial Cells for Angiogenesis, Science 275: 964-966 (1997)); however in diabetes these cells often become dysfunctional (Chen, Y. H., et al., High Glucose Impairs Early and Late Endothelial Progenitor Cells by Modifying Nitric Oxide-Related but Not Oxidative Stress-Mediated Mechanisms, Diabetes 56: 1559-1568 (2007)). The inventor previously discovered that blocking TGF-β1 in human CD34⁺ endothelial progenitor cells (EPCs) corrects many aspects of their dysfunctional behavior (Bhatwadekar, Ashay D., et al., Transient Inhibition of Transforming Growth Factor-B1 in Human Diabetic CD34+ Cells Enhances Vascular Reparative Functions, Diabetes, 119 (2010)). As formerly determined by the inventor, transient inhibition of TGF-β1 enhances vascular reparative function of human CD34⁺ cells isolated from diabetics (Bhatwadekar et al, 2010). PAI-1 is the major gene product of TGF-β1 activation. PAI-1 levels are increased in diabetes, atherosclerosis and obesity (Pandolfi, A., et al., Plasminogen Activator Inhibitor Type 1 is Increased in the Arterial Wall of Type II Diabetic Subjects, Arterioscler. Thromb. Vasc. Biol. 21:1378-1382 (2001)). PAI-1 blocks plasmin generation by inhibiting activities of serine proteinasesurokinase plasminogen activator (uPA) and tissue type plasminogen activator (t-PA). Plasmin is a key enzyme in extracellular matrix (ECM) degradation. PAI-1 is a single chain glycoprotein (50 kDa molecular weight) present in blood in very low concentrations. Its expression is influenced by various cytokines and growth factors and its activity is regulated on the transcriptional level (Binder, B. R., et al., Plasminogen Activator Inhibitor 1: Physiological and Pathophysiological Roles. News Physiol. Sci. 17:56-61 (2002)). Transcription of the PAI-1 gene is modulated by hypoxia ((114 Uchiyama, Tsuyoshi 2000)). PAI-1 also inhibits smooth muscle cell migration by blocking binding of integrin αᵥβ₃ to vitronectin (Stefansson, S., et al., The Serpin PAI-1 Inhibits Cell Migration by Blocking Integrin Alpha V Beta 3 Binding to Vitronectin. Nature, 383:441-443 (1996)).

PI3K/Akt, the signaling pathway mediating cell survival, proliferation, and migration (Cantley, L. C., et al., The Phosphoinositide 3-Kinase Pathway. Science 296:1655-1657 (2002)) negatively regulates PAI-1 expression in vascular endothelial cells (Mukai, Y., et al., Phosphatidylinositol 3-kinase/protein kinase Akt Negatively Regulates Plasminogen Activator Inhibitor Type 1 Expression in Vascular Endothelial Cells. Am. J. Physiol. Heart Circ. Physiol. 292:H1937-1942 (2007)). Inhibition of PAI-1 using PAI-1 selective antibody increased migration of human CD34⁺ across rat endothelial cell monolayer (Xiang, G., et al., Down-regulation of plasminogen activator inhibitor 1 expression promotes myocardial neovascularization by bone marrow progenitors. J. Exp. Med. 200:1657-1666 (2004)).

The 4G/5G promoter allele of the PAI-1 gene is strongly linked to type 2 diabetes (Nagi, D. K., et al., Diabetic retinopathy, promoter (4G/5G) polymorphism of PAI-1 gene, and PAI-1 activity in Pima Indians with type 2 diabetes. Diabetes Care 20:1304-1309 (1997)). Increased levels of PAI-1 are accompanied by increased levels of urokinase and metalloprotease enzymes in human diabetic microvascular membranes (Das, A., et al., Human diabetic neovasuclar membranes contain high levels of urokinase and metalloprotease enzymes. Invest. Opthalmol. Vis. Sci. 40:809-813 (1999)). PAI-1 expression is increased in retina with oxygen-induced retinopathy (Basu, A., et al., Plasminogen Activator Inhibitor-1 (PAI-1) Facilitates Retinal Angiogenesis in a Model of Oxygen-Induced Retinopathy. Invest. Oopthalmol. Vis. Sci. 50:4974-4981 (2009)). Previously, the inventor showed that PAI-1 is over expressed in capillaries of diabetic patients with non-proliferative diabetic retinopathy (Grant, M. B., et al., Plasminogen activator inhibitor-1 over expression in non-proliferative diabetic retinopathy. Exp. Eye Res. 63:233-244 (1996)) and PAI-1^{-/-} animals are protected from development of diabetic retinopathy (Grant, M. B., et al., Plasminogen activator inhibitor (PAI-1) over expression in retinal microvessels of PAI-1 transgenic mice. Invest. Opothalmol. Vis. Sci. 41:2296-2302 (2000)).

It is readily apparent from these studies, collectively, that PAI-1 has a central role in critical aspects of diabetes-related vascular pathology. The inventor has identified whether the beneficial effects of TGF-β1 blockade on EPC function are mediated by PAI-1 inhibition and whether blocking PAI-1 alone corrects diabetes associated dysfunction of EPCs.

The present invention is based on and is a further development of the inventors' discovery that transient blocking of TGF-β in EPCs enhances the ability of such cells to proliferate, migrate and home into areas of injury. The inventor has discovered that treatment of stem cells, particularly, HSCs increases their homing ability to vascular lesions, and thus increases the reparative potential of the treated HSCs. Endothelial precursor cells have the ability to promote vascular repair. The approach outlined herein includes identifying the effect of inhibiting PAI-1 and the effect of this blockage on the recruitment of diabetic as well as healthy CD34⁺ cells to sites of retinal injury. In addition, this approach also focuses on the effect inhibiting PAI-1 has on the correction of defective repair in the diabetic CD34⁺ cells. The inhibition of PAI-1 in diabetic CD34⁺ cells ex vivo enhances repair following vascular damage. This inhibition treatment enhances the vascular repair potential, which is applicable to all vessels. This finding has a profound impact on disease states associated with vascular dysfunction such as for example, ischemic heart disease and diabetic vascular complications. Attempts are often made to replace traditional approaches for alleviating tissue ischemia (e.g., stents, angioplasty, or vessel grafts) with cell therapy, as autologous cell therapy is limited in diabetic patients because of dysfunctional cells. The inventor has determined that cytostatic activity of TGF-B1 in CD34⁺ cells is mediated largely through PAI-1, and that blocking PAI-1 corrects multiple defects in CD34⁺ cells from type 2 diabetic patients. Inhibition of PAI-1 provides a promising therapeutic strategy for restoring vascular reparative function in many cells, and particularly in diabetic CD34⁺ cells. According to one embodiment of the invention, repair of coronary vessels following myocardial infarction is achieved by administration of treated stem cells. In another embodiment, cerebral vessels are repaired following stroke. In addition, injured peripheral vascular beds are repaired by administration of treated cells.

Many of the embodiments of the subject invention make reference to particular methods of inhibiting expression. The subject invention is not to be limited to any of the particular methods described. One such method includes siRNA (small interfering/short interfering/silencing RNA). SiRNA most often is involved in the RNA interference pathway where it interferes with the expression of a specific gene. In addition to its role in the RNA interference pathway, siRNA also act in RNA interference-related pathways, e.g., as an antiviral mechanism or in shaping the chromatin structure of a genome.

Another method by which to inhibit expression and to inhibit the expression of PAI-1 in particular is shRNA. ShRNA (short hairpin or small hairpin RNA) refers to a sequence of RNA that makes a tight hairpin turn and is used to silence gene expression via RNA interference. It uses a vector introduced into cells and a U6 or H1 promoter to ensure that the shRNA is always expressed. The shRNA hairpin structure is cleaved by cellular machinery into siRNA which is then bound to the RNA-induced silencing complex. This complex binds to and cleaves mRNAs which match the siRNA that is bound to it.

PAI-1 can also be blocked by subjecting procured cells to an antibody specific to PAI-1. An antisense nucleotide may also be used to block or inhibit expression, in particular, the expression of PAI-1. Expression may also be inhibited with the use of a morpholino oligomer or phosphorodiamidate morpholino oligomer (PMO). PMOs are an antisense technology used to block access of other molecules to specific sequences within nucleic acid. PMOs are often used as a research tool for reverse genetics, and function by knocking down gene function. This is achieved by preventing cells from making a targeted protein or by modifying splicing of pre-mRNA. One embodiment of the subject invention pertains to a method of treating vascular lesions in a subject in need thereof. The term “subject” as used herein refers to a human or a non-human mammal. Non-human mammals include, but are not limited to, rodents such as rats and mice, cats, dogs, horses, cattle, goats, sheep or pigs. The method involves procuring hematopoietic stem cells from the subject to obtain procured hematopoietic stem cells. The procured hematopoietic stem cells are treated, ex vivo, by blocking activity of PAI-1 in the cells. Examples of PAI-1 blocking agents are disclosed in U.S. Pat. Nos. 6,869,795, 6,333,408, and US Pub. No. 2008/0019910A1. The treated hematopoietic stem cells are administered to the subject. In a specific embodiment, PAI-1 is blocked by subjecting procured cells with an antibody specific to PAI-1. Specific examples of antibodies useful in accordance with the teachings herein are taught in U.S. Patent Pub. No. 2007/0081988A1. In another specific embodiment, PAI-1 is blocked by an antisense nucleotide. Specific examples of anti-sense oligomers useful in accordance with the teachings herein are disclosed in U.S. Patent Pub. No. 2004/0224912A1. The method in another embodiment is used to treat the patient, wherein the vascular lesions are associated with choroidal neovascularization. Choroidal neovascularization (CNV) relates to the creation of new blood vessels in the choroid layer of the eye. CNV can be used as a reparative technique following damage resulting from degenerative maculopthy wet AMD (age-related macular degeneration).

As provided by the methods of the invention herein, the term “administering”, “administer” or “administration” with respect to delivery of cells to a subject refers to injecting one or a plurality of cells with a syringe, inserting the stem cells with a catheter or surgically implanting the stem cells. In certain embodiments, the stem cells are administered into a body cavity fluidly connected to a target tissue. In other embodiments, the stem cells are inserted using a syringe or catheter, or surgically implanted directly at the target tissue site. In other embodiments, the stem cells are administered systemically (e.g., parenterally). In other specific examples, stem cells are administered by intraocular delivery, intramuscular delivery, subcutaneous delivery or intraperitoneal delivery.

As provided by the methods of the invention herein, the term “administering”, “administer” or “administration” with respect to delivery of a PAI-1 blocking agent to a subject refers to parenteral administration, intraperitoneal, intramuscular, intraocular administration including transcleral administration, and intravitreal injection; transdermal administration, oral administration, intranasal administration, direct delivery to a target site or delivery to a body cavity in fluid communication with a target site.

As used herein, the term “enhancing repair of a vessel lesion” refers to an improvement in the state of a lesion in blood vessels in the body. Improvement in the state may involve partial or full healing of the lesion. Healing of the vessel lesion may include remodeling of the wounded tissue at the lesion and surrounding tissue.

As used herein, the terms “antisense oligonucleotide” and “antisense oligomer” are used interchangeably and refer to a sequence of nucleotide bases and a subunit-to-subunit backbone that allows the antisense oligonucleotide or oligomer to hybridize to a target sequence in an RNA by Watson-Crick base pairing, to form an RNA:oligomer heteroduplex within the target sequence. The oligomer may have exact sequence complementarity to the target sequence or near complementarity. Such antisense oligomers may block or inhibit translation of the mRNA containing the target sequence, or inhibit gene transcription, may bind to double-stranded or single stranded sequences, and may be said to be “directed to” a sequence with which it hybridizes.

The term “coadministering” or “concurrent administration”, when used, for example with respect to PAI-1 blocking agent and a sample of treated cells, refers to administration of the agent and the cells such that both can simultaneously achieve a physiological effect. The agent and the cells, however, need not be administered together. In certain embodiments, administration of one can precede administration of the other, however, such coadministering typically results in both agent and cells being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

In a further embodiment, a method of diminishing diabetic retinopathy in a subject is provided. The method includes administering hematopoietic stem cells treated with a PAI-1 blocking agent to the subject. The administering may include parenterally injecting the cells, in a specific embodiment, or in an alternative embodiment, by intraoptic injection.

In another embodiment, hematopoietic stem cells may be obtained from a patient in need of transplantation, (e.g., a patient having a stroke or myocardial infarction event, a patient suffering from CNV, a patient suffering from atherosclerosis, a diabetic patient, or any other patient having a vessel lesion or risk of vessel lesion); enriched, treated in vitro (ex vivo) using the methods described herein, and returned to the patient.

In practicing a specific embodiment of the invention, hematopoietic stem cells may be treated in vitro (ex vivo) with one or more oligonucleotide antisense to a nucleic acid sequence that is preferentially expressed in stem cells, followed by administration to a subject. The subject may be the same individual from whom the stem cells were obtained (autologous transplantation) or a different individual (allogeneic transplantation). In allogeneic transplantation, the donor and recipient are matched based on similarity of HLA antigens in order to minimize the immune response of both donor and recipient cells against the other. The administration of cells subjected ex vivo to a PAI-1 blocking agent to the subject may be co-administered with a therapeutically effective amount of a PAI-1 blocking agent to the subject. This method may be carried out for patients in need, such patients suffering from conditions such as diabetes, nephropathy, diabetic neuropathy, choroidal neovascularization, myocardial infarction, stroke, and other potential conditions rendering a patient in need of such treatment.

In one aspect, the invention is directed to methods of modifying the development of hematopoietic stem cells, by obtaining a population of HSCs and exposing them ex vivo to one or more nuclease-resistant antisense oligomers having high affinity to a complementary or near-complementary nucleic acid sequence preferentially expressed in stem cells. In another aspect, a population of HSCs is exposed to an anti-PAI-1 antibody.

In one aspect, once extracted and enriched, stem cells, e.g., HSC, may be cultured ex vivo in the presence of one or more cytokines and one or more antisense oligomers and/or antibodies described herein. Such an antisense oligomer, and/or anti anti-PAI-1-treated hematopoietic stem cell composition finds utility in repairing, enhancing repair of vascular lesions.

Examples of cytokines for such ex vivo culture include, but are not limited to IL-3, IL-6, SCF and TPO. A hematopoietic stem cell population for use in the methods of the invention is typically both human and allogeneic, or autologous.

Exemplary antisense oligomers target one or more of an EVI-1 zinc finger gene, a serum deprivation response (SDR) gene, a multimerin gene, a tissue transglutaminase gene, an FE65 gene, a RAB27 gene, a Jagged2 gene, a Notch1 gene, a Notch2 gene and a Notch3 gene.

Once a large number of cells, i.e., cells of a particular lineage, are obtained, the cells can be used immediately or frozen in liquid nitrogen and stored for long periods of time, using standard conditions, such that they can later be thawed and used, e.g., for administration to a patient. The cells will usually be stored in 10% DMSO, 50% fetal calf serum (FCS), and 40% cell culture medium.

In another aspect, the invention is directed to methods of modifying the development of stem cells in vivo in a patient in need thereof, by administering to the patient a therapeutically effective amount of an antisense oligonucleotide-containing composition, where the antisense oligomer modulates the expression of a gene product preferentially expressed in stem cells.

Such in vivo antisense oligomer administration may also be effective to improve the therapeutic outcome of the subject by effecting an enhancement of repair potential of endogenous untreated stem cells, or stem cells which have undergone, ex vivo, treatment and then administered to the subject.

In one example, the antisense oligonucleotide composition is administered at a concentration and for a period sufficient to increase the population of progenitor cells. It will be understood that in vivo administration of such an antisense oligomer to a subject using the methods of the invention can provide a means to increase the population of lineage committed progenitor cells and their progeny in the peripheral circulation of the subject, and/or effect a slowing or diminution of the growth of cancer cells or a solid tumor, or a reduction in the total number of cancer cells or total tumor burden, dependent upon, (1) the duration, dose and frequency of antisense administration, (2) the one or more antisense oligomers used in the treatment; and (3) the general condition of the subject.

It is appreciated that any methods which are effective to deliver the PAI-1 blocking agent to hematopoietic stem cells or to introduce the agent into the bloodstream are also contemplated.

Transdermal delivery of PAI-1 blocking agent may be accomplished by use of a pharmaceutically acceptable carrier adapted for e.g., topical administration. One example of morpholino oligomer delivery is described in PCT patent application WO 97/40854, incorporated herein by reference.

In one specific embodiment, the PAI-1 blocking agent, contained in a pharmaceutically acceptable carrier, and delivered orally. In a further aspect of this embodiment, a PAI-1 blocking agent is administered at regular intervals for a short time period, e.g., daily for two weeks or less. However, in some cases the PAI-1 blocking agent is administered intermittently over a longer period of time.

Typically, one or more doses of PAI-1 blocking agent are administered, generally at regular intervals for a period of about one to two weeks. Preferred doses for oral administration are from about 1 μg agent/patient to about 25 mg oligomer/patient (based on an adult weight of 70 kg). In some cases, doses of greater than 25 mg blocking agent patient may be necessary. For IV administration, the preferred doses are from about 0.05 mg agent/patient to about 10 mg agent/patient (based on an adult weight of 70 kg).

The antisense compound is generally administered in an amount sufficient to result in a peak blood concentration of at least 200-400 nM blocking agent.

In one example, the method includes administering to a subject, in a suitable pharmaceutical carrier, an amount of an antisense agent effective to inhibit expression of a nucleic acid target sequence of interest.

It follows that a blocking agent composition may be administered in any convenient vehicle, which is physiologically acceptable. Such blocking agent composition may include any of a variety of standard pharmaceutically accepted carriers employed by those of ordinary skill in the art. Examples of such pharmaceutical carriers include, but are not limited to, saline, phosphate buffered saline (PBS), water, aqueous ethanol, emulsions such as oil/water emulsions, triglyceride emulsions, wetting agents, tablets and capsules. It will be understood that the choice of suitable physiologically acceptable carrier will vary dependent upon the chosen mode of administration

In some instances liposomes may be employed to facilitate uptake of the blocking agent into cells. (See, e.g., Williams, 1996; Lappalainen, et al., 1994; Uhlmann, et al., 1990; Gregoriadis, 1979.) Hydrogels may also be used as vehicles for antisense oligomer administration, for example, as described in WO 93/01286. Alternatively, the blocking agent may be administered in microspheres or microparticles. (See, e.g., Wu and Wu, 1987).

Sustained release compositions are also contemplated within the scope of this application. These may include semi permeable polymeric matrices in the form of shaped articles such as films or microcapsules.

It will be understood that the effective in vivo treatment regimen of the blocking agent in the methods of the invention will vary according to the frequency and route of administration as well as the condition of the subject under treatment. Accordingly, such in vivo therapy will generally require monitoring by tests appropriate to the condition being treated and a corresponding adjustment in the dose or treatment regimen in order to achieve an optimal therapeutic outcome.

The efficacy of a given therapeutic regimen involving the methods described herein, may be monitored, e.g., by conventional FACS assays for the phenotype of cells in the circulation of the subject under treatment in order to monitor changes in the numbers of cells of various lineages (e.g., lineage committed progenitor cells and their progeny) in the peripheral circulation of the subject in response to such treatment.

Phenotypic analysis is generally carried out using monoclonal antibodies specific to the cell type being analyzed, e.g., neutrophils, platelets, lymphocytes, erthryrocytes or monocytes. The use of monoclonal antibodies in such phenotypic analyses is routinely employed by those of skill in the art for cellular analyses. Monoclonal antibodies specific to particular cell types are commercially available.

Hematopoietic stem cells are characterized phenotypically as detailed above. Such phenotypic analyses are generally carried out in conjunction with biological assays for each particular cell type of interest, for example (1) hematopoietic stem cells (LTCIC, cobblestone forming assays, and assays for HPP-CFCs), (2) granulocytes or neutrophils (clonal agar or methyl cellulose assays wherein the medium contains G-CSF or GM-CSF), (3) megakaryocytes (clonal agar or methyl cellulose assays wherein the medium contains TPO, IL-3, IL-6 and IL-11), and (4) erythroid cells (clonal agar or methyl cellulose assays wherein the medium contains EPO and SCF or EPO, SCF and IL-3).

It will be understood that the exact nature of such phenotypic and biological assays will vary dependent upon the condition being treated and whether the treatment is directed to enhancing the population of hematopoietic stem cells or the population of cells of a particular lineage or lineages.

In cases where the subject has been diagnosed as having a particular type of lesion, the status of the lesion is also monitored using diagnostic techniques appropriate to the type of lesion under treatment to determine if repair of the lesion has progressed.

It is noted that PAi-1 is also known in the art as serpine-1.

### Example 1

### Type2 Diabetes is Associated with Increased Level of Plasma PAI-1 Compared to Type 1, Although TGF-β1 Level was Similar

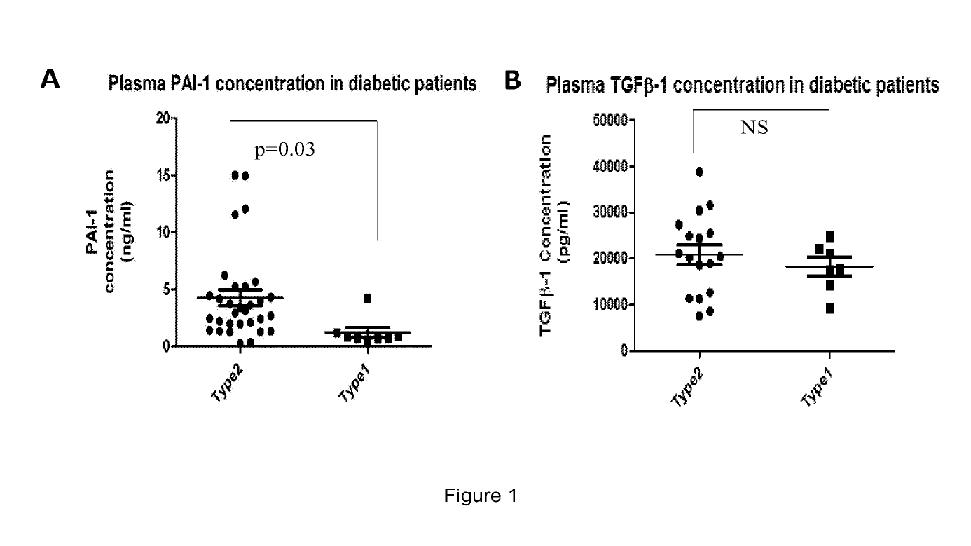

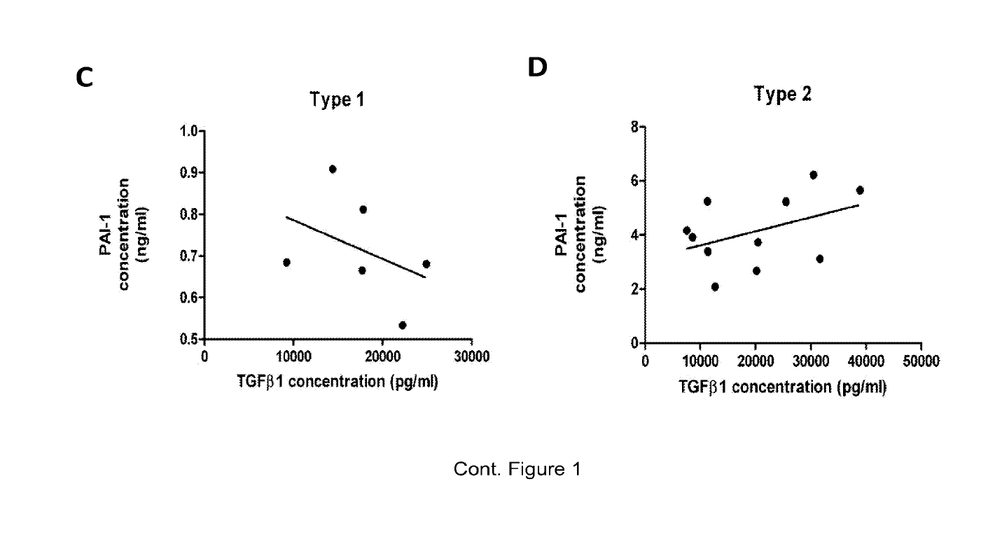

As plasma levels of PAI-1 and TGF-β1 may also have an effect in the CD34⁺ function plasma levels of PAI-1 and TGF-β1 were also measured in the patient population are shown in FIGS. 1A and 1B. Turning to the Figures, FIG. 1A-D provides a graphical illustration of a comparison of plasma PAI-1 and TGF-β1 concentrations with Type 1 and Type 2 diabetes. It is apparent from the Figures that Type 2 diabetes is associated with increased level of plasma PAI-1 compared with Type 1 diabetes. TGF-β1 levels appear similar in both cases.

Plasma PAI-1 level was higher in type 2 diabetics compared to type 1 diabetics (n=20 for type 2, n=8 for type 1, p=0.03 FIG. 1A). However, TGF-β1 levels were similar in both groups (n=17 for type 2, n=7 for type 1 see FIG. 1B). When plasma TGF-β1 levels were compared with PAI-1 in type 2 diabetics (FIG. 1C), a positive correlation was found (with a coefficient of correlation 0.44). In contrast, a negative correlation was found between PAI-1 and TGF-β1 in type 1 diabetes (FIG. 1D with a correlation coefficient −0.39).

### Example 2

### Diabetic Patients Protected from Development of Microvascular Complications Exhibit Reduced Expression of PAI-1 in CD34⁺ Cells

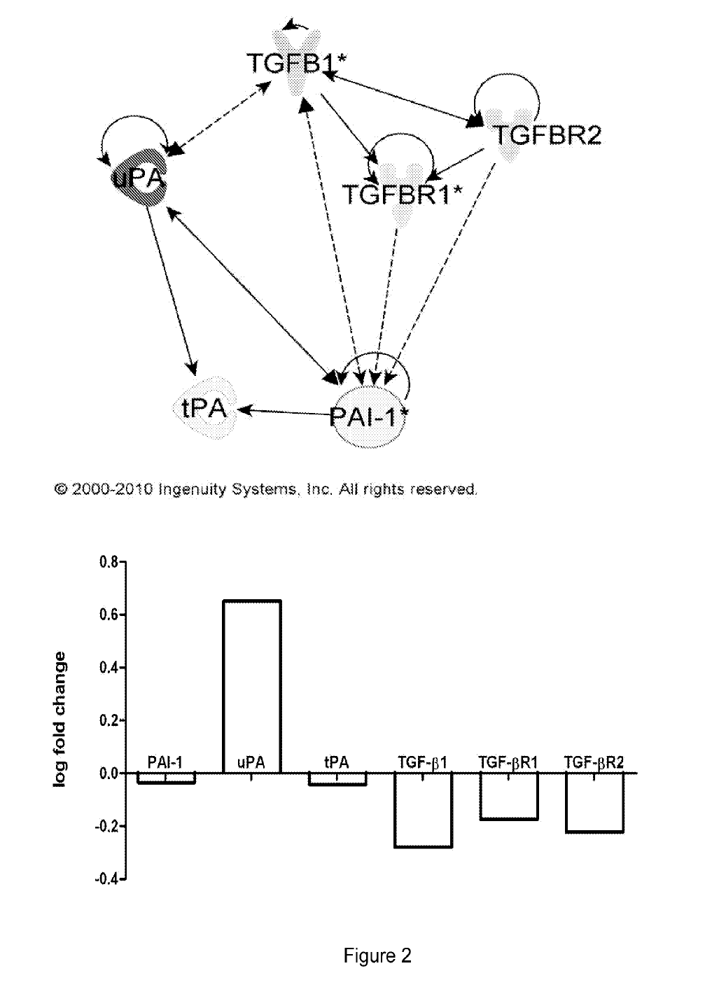

Previously it was shown that activity and antigen levels of uPA are down-regulated in patients having non-insulin dependent diabetes mellitus and tPA, and PAI-1 levels were up-regulated. In order to determine whether similar signature was found in the CD34⁺ cells isolated from the patient group herein microarray was conducted on CD34⁺ cells obtained from a) diabetic patients with microvascular complications (n=5); b) diabetic patients without microvascular complications (n=5) and c) healthy age-matched controls (n=5). The data was analyzed using Ingenuity pathway analysis software. Referring to FIG. 2A, a pathway map showing alteration in gene expressions in patients protected from development of microvascular complications compared to the diabetic patients who developed microvascular complications is provided. FIG. 2B provides a bar chart showing log fold change for respective gene. It is clearly shown herein that in this patient population, TGF-β1, TGF-βR1, TGF-βR2, PAI-1 and tPA (tissue plasminogen activator) are down-regulated whereas uPA (urokinase plasmonigen activator) is up-regulated. This data represents that the gene expression of the CD34⁺ cells isolated from the protected patients shows a similar trend.

Inventors identified that diabetic patients protected from development of microvascular complications would have more robust endothelial progenitors and be able to elicit a better repair response than endothelial progenitors from diabetic that manifest microvascular complications. As abovementioned, the Inventors selected a group of a unique diabetic population, individuals without microvascular complications (n=5) despite greater than 40 years of diabetes with largely poor metabolic control throughout this time. These were compared to diabetic patients with microvascular complications but that were matched for sex, age, duration of diabetes and glucose control (n=5) as well as to healthy age/sex matched controls (n=5) (Table 1).

In reference to FIG. 2, it is demonstrated that diabetic patients protected from development of microvascular complications exhibit reduced expression of PAI-1 and increased expression of uPA in CD34+ cells. The pathway map provides an expression profile for patients protected from development of microvascular complications compared to those with complications (FIG. 2A). In CD34+ endothelial progenitor cells from protected patients, TGF-β1, TGF-βR1, TGF-βR2, PAI-1 and tPA (tissue plasminogen activator) were down-regulated whereas uPA (urokinase plasmonigen activator) was up-regulated, suggesting reduced activation of the TGF-β1-PAI-1 system (FIG. 2B). FIG. 2 represents Diabetic patients protected from development of microvascular complications exhibit reduced expression of PAI-1 and increased expression of uPA in CD34+ cells. Microarray was conducted on CD34+ cells obtained from diabetic patients with microvascular complications (n=5), diabetic patients without microvascular complications (n=5), healthy age matched controls (n=5). The data was analyzed using Ingenuity pathway analysis software. The pathway map of FIG. 2A shows alteration in gene expressions in patients protected from development of microvascular complications compared to the diabetic patients who developed microvascular complications. FIG. 2B provides a bar chart showing log fold change for respective gene.

### Example 3

### Increased Secreted Level of PAI-1 from Type 2 Diabetic CD34⁺ Cells

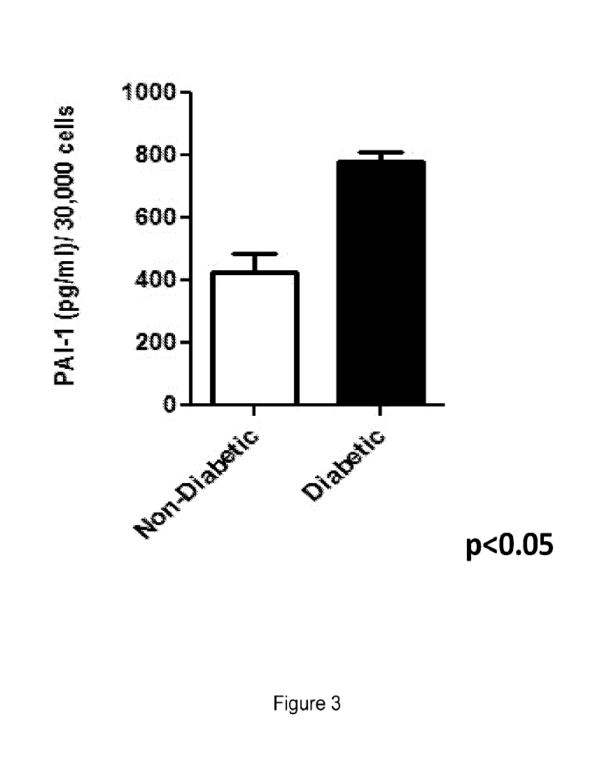

The CD34⁺ cells from both diabetic and non-diabetic individuals were characterized in terms of their ability to release PAI-1 in the conditioned media (CM). As shown in FIG. 3, the diabetic cells showed a significantly higher level of secreted PAI-1 in the conditioned media as compared to the non-diabetic cells (p<0.05).

### Example 4

### TGF-β1 Regulates PAI-1 Expression in Both Diabetic and Non-Diabetic CD34⁺ Cells

The inventor has previously demonstrated that blocking expression of TGF-β1 in diabetic CD34⁺ cells corrected diabetes-associated reductions in migration, NO generation, and in vivo homing (Bhatwadekar A 2010). PAI-1 is the major gene product of TGF-β1 pathway activation.

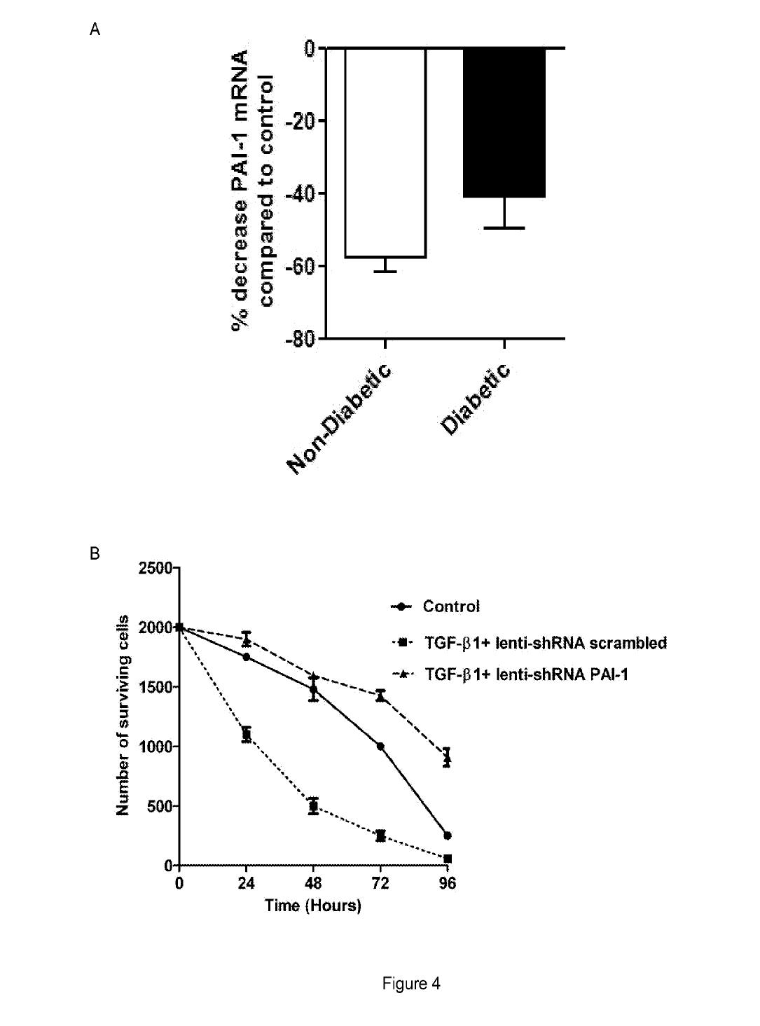

PAI-1 expression in diabetic and normal CD34⁺ cells was examined following TGF-β1 PMO or scrambled PMO treatment. Both diabetic and non-diabetic CD34⁺ cells treated with TGF-β1 PMO demonstrated decreased PAI-1 mRNA expression compared to cells treated with scrambled PMO (n=10 for diabetic and n=3 for non-diabetic, p<0.001 for diabetic and p=0.05) (see FIG. 4A).

### Example 5

### PAI-1 Blockade Eliminates the Inhibitory Effect of TGF-β1 on CD34⁺ Cells and Increased Proliferation of Healthy and Diabetic CD34⁺ Cells Following Continuous Exposure to Growth Factors

TGF-β1 inhibits proliferation of progenitor cells and is largely responsible for maintaining stem cells quiescence. To determine whether the inhibitory effect of TGF-β1 on cells survival was mediated by PAI-1, CD34⁺ cells were exposed herein to either lentivirus expressing PAI-1 shRNA or scrambled shRNA. Following lenti virus infection, cells were treated with recombinant human TGF-β1 (1 ng/ml) for 24 hours without growth factors, and cell viability was determined over 72 hours. As shown in FIG. 2A, the cells without addition of growth factors showed a marked decrease in cell number even at 24 hours, and this decrease became more pronounced over time. The number of surviving cells that were exposed to lentivirus expressing scrambled shRNA followed by treatment with TGF-β1 also decreased over time. In contrast, when the cells were exposed to lentivirus expressing PAI-1 shRNA followed by TGF-β1 addition, there was a significant increase in the number of surviving cells even in the absence of growth factors (p<0.001 for TGF-β1+PAI-1shRNA compared to TGF-β1+ scrambled shRNA), suggesting that TGF-β1 mediates the inhibitory effects on CD34⁺ cells survival by PAI-1. The CD34⁺ cells also express low-density lipoprotein receptor-related protein 1 (LRP-1), the putative receptor for PAI-1.

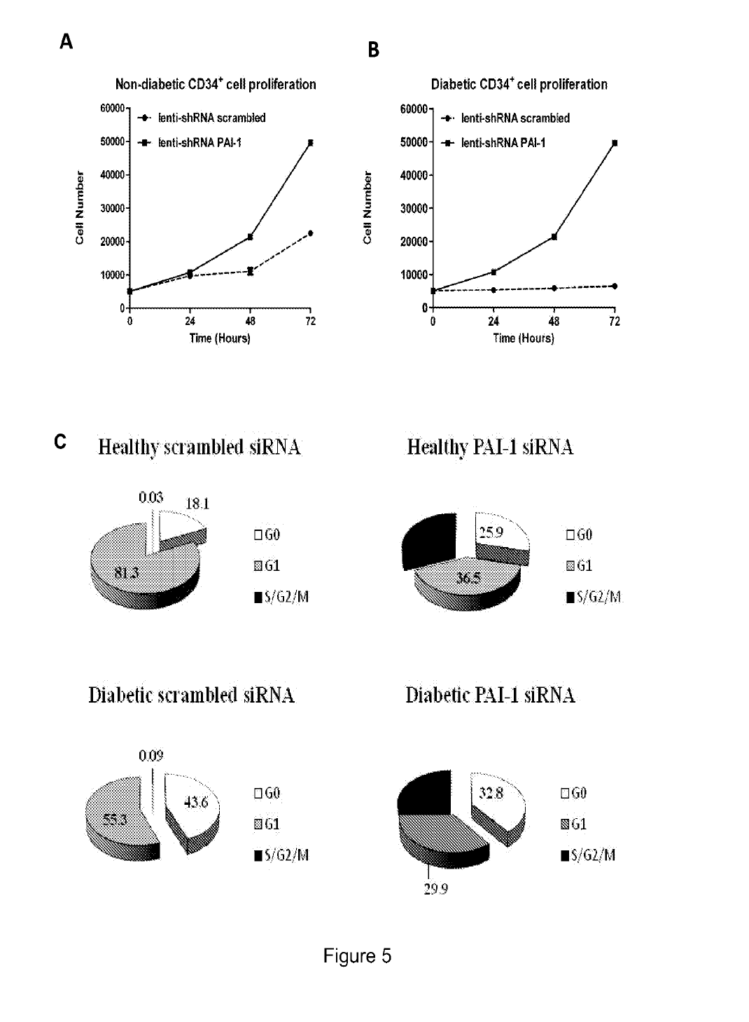

The effect of PAI-1 blockade on the proliferative capacity of CD34⁺ without the growth inhibitor TGF-β1, but in the presence of growth promoting factors was tested next. Control and diabetic CD34⁺ cells were infected with either lentivirus expressing PAI-1 shRNA or scrambled shRNA and exposed to growth factors for 72 hours. Non-diabetic CD34⁺ cells (FIG. 2B) proliferated following PAI-1 blockade to a greater degree than cells treated with lenti shRNA control. In contrast, diabetic CD34⁺ cells did not proliferate even in the presence of growth factors; however, PAI-1 blockade increased the proliferation rate of the diabetic cells to the level of non-diabetic cells (FIG. 2C). Particularly relevant to the use of CD34⁺ cells for cell therapy is the requirement to ex vivo expand the cells prior to re-administering them to a patient. Ideally a strategy is needed that would allow expansion of the cells without their differentiation. To determine whether PAI-1 blockade was associated with stimulating CD34⁺ cell proliferation, the number of cells that were in G0 and in G1 at days 5 and 7 were examined. As shown in the figure, following PAI-1 siRNA treatment, in both non-diabetics and diabetics, fewer cells were in G0 and more cells were in the active stage of the cell cycle, suggesting that reducing PAI-1 facilitated the transition of cells through the cell cycle. Moreover, it is ideal to minimize the exposure of progenitors to growth factors, as this prompts differentiation and limits the ex vivo expansion potential of these cells. CD34⁺ cells were thus treated with PAI-1 siRNA and then exposed to growth factors for only 24 hours followed by withdrawal of all growth factors. Compared to control siRNA-treated cells, the inhibition of PAI-1 allowed a greater number of cells to survive in the absence of growth factors for 6 days (see FIG. 5C) (78.5% increase compared to control siRNA) (data not shown).

### Example 6

### PAI-1 Blockade Improved Migration of Both Healthy and Diabetic CD34⁺ Cells in Response to the Chemo-Attractant SDF-1α

Diabetic CD34⁺ cells demonstrate reduced migratory prowess and PAI-1 has been shown to influence cell migration. The effect of PAI-1 on the migratory ability of CD34⁺ cells was examined using SDF-1α as the chemoattractant.

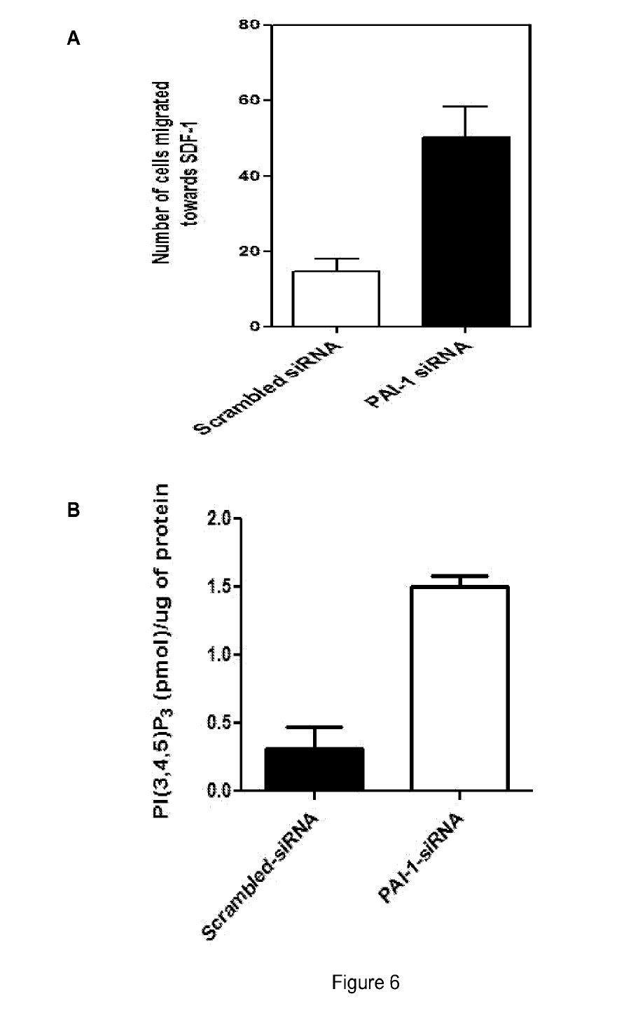

Diabetic CD34⁺ cells were treated with either PAI-1 siRNA or scrambled siRNA and 24 hours later their migration to SDF-1α (100 nM) was examined. Cells infected with PAI-1 siRNA demonstrated greater migratory response compared to the cells treated with scrambled siRNA (see FIG. 6A) suggesting that reducing PAI-1 in the diabetic cells improved their migratory ability in vitro.

### Example 7

### PAI-1 Blockade Results in Increased PI3K/AKT Activity in CD34⁺ Cells

To examine the signaling pathway by which blocking PAI-1 induces increased survival, proliferation, and migration potential of the CD34⁺ cells, an assay was used for phosphatidylinositol 3-kinase (PI3K) activity involving determination of the conversion of PI(3,4,5)P₃ to PI(4,5)P₂. Blocking PAI-1 stimulated PI3K activity significantly compared to the activity when the cells were treated with scrambled siRNA (p<0.05) (see FIG. 6B).

As the PI3K-AKt pathway is also associated with NO generation, NO generation was measured by measuring DAF-FM fluorescence and also quantified cGMP production in the healthy and diabetic CD34⁺ cells. Blocking PAI-1 had no effect in NO generation and also failed to improve cGMP production in healthy CD34⁺ cells (data not shown), suggesting that blocking PAI-1 will be beneficial for diabetic, but not for healthy patients.

### Example 8

### Mir-146a can Reduce PAI-1 mRNA Expression in the CD34⁺ Cells

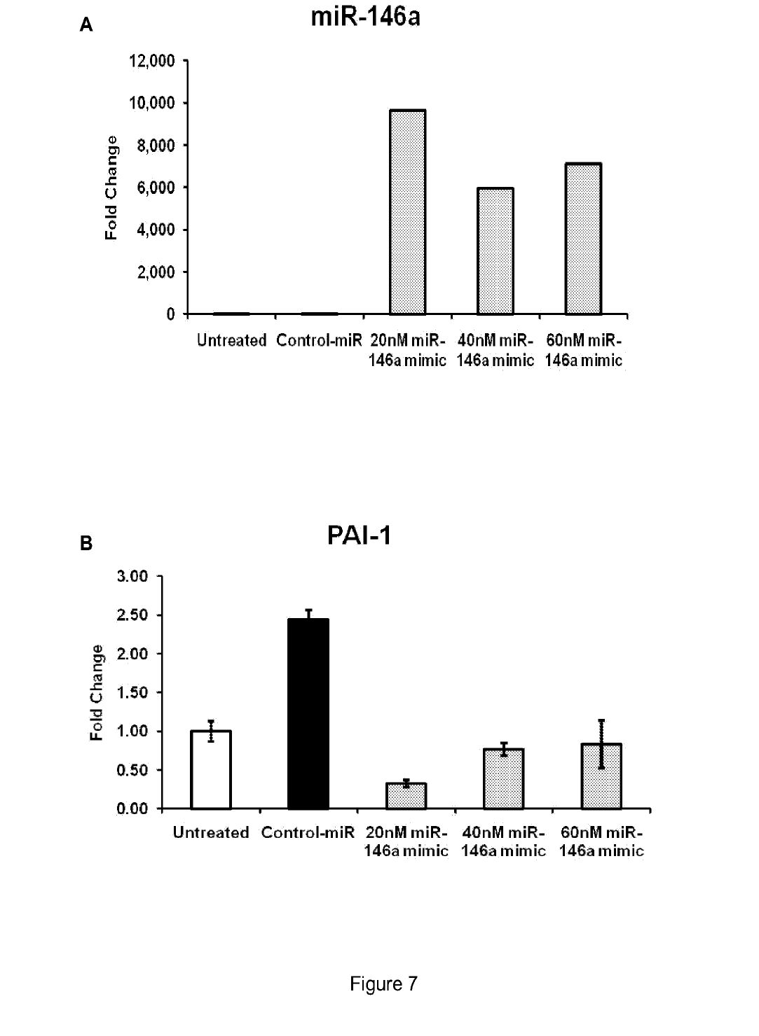

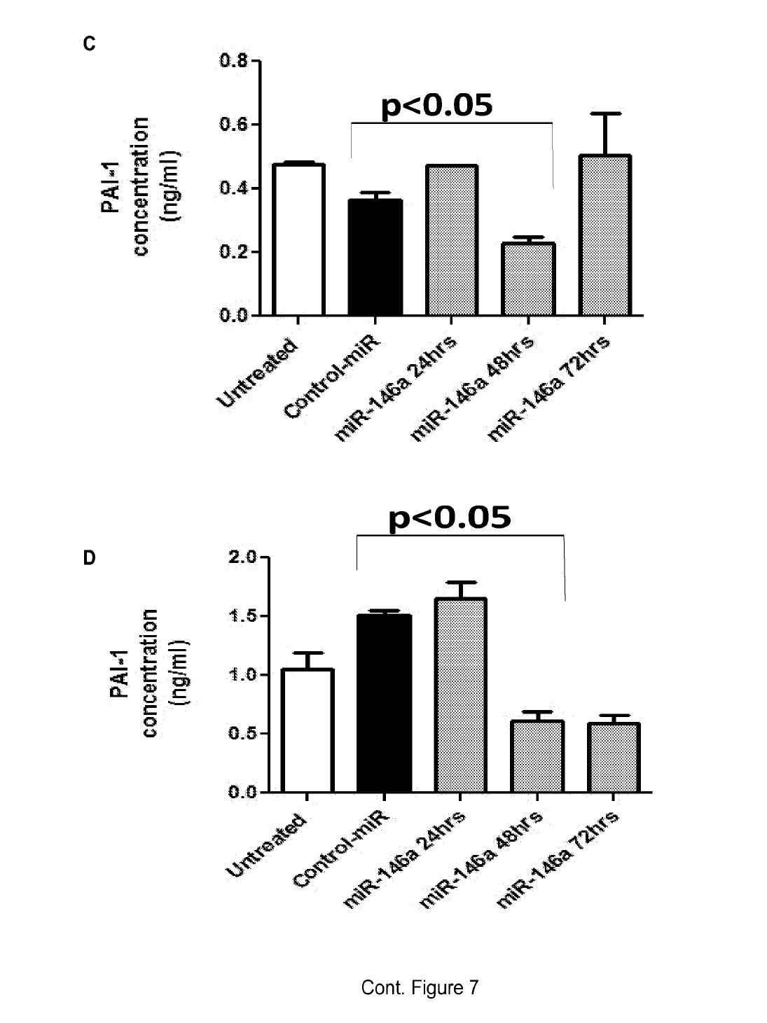

There was a desire to investigate the role of microRNA in the expression of PAI-1 and to regulate the expression of PAI-1 by regulating microRNA. MiR-146a was selected as it has been found to modulate PAI-1 expression in human trabecular meshwork cell. To determine the direct role of miR-146a in PAI-1 mRNA expression in the progenitor cells, healthy CD34⁺ cells were transfected with 20 nM, 40 nM, and 60 nM miR-146a mimic for 24 hours. A significant increase in miR-146a expression in miR-146a transfected cells versus the untreated cells was found, therefore confirming successful transfection (see FIG. 7A). As shown in FIG. 7B, cells transfected with miR-146a mimic produced ˜7 times less PAI-1 transcripts in comparison to untreated cells 24 hours after transfection, suggesting that up-regulation of miR-146a reduces the PAI-1 mRNA expression in CD34⁺ cells.

Overexpressing miR-146a also reduced secreted level of PAI-1 in the CM of both non-diabetics (see FIG. 7C) and diabetics (see FIG. 7D), although for diabetics, the concentration of miR-146a mimic used was higher, as the basal level of PAI-1 was higher.

Therapeutic revascularization with autologous endothelial progenitor cells holds promise to prevent tissue damage and restore blood flow in diabetics who are not ideal candidates for standard revascularization procedures due to diffuse vascular disease or failed previous revascularization. However, while novel therapy is needed in diabetic patients, the autologous approach is limited due to endothelial progenitor cell dysfunction (Caballero, Sergio et al, 2007; Busik, Julia V. et al, 2009; Loomans, C. J. M. et al, 2004; Thum, Thomas et al, 2007). Specifically, endothelial progenitors isolated from diabetic individuals demonstrate reduced proliferation, migration, and differentiation into endothelial cells (Tepper, Oren M. et al, 2002; Segal, M. S. et al, 2006). Exposure to high concentrations of glucose reduces endothelial eNOS expression in these cells [Chen, Y. H. et al, 2007]. Progenitors for db/db mice show reduced expression of eNOS and phospho-eNOS. Consistent with this finding, it has been shown herein that human diabetic CD34⁺ cells have reduced NO bioavailability associated with decreased migration that can be restored through exposure to NO donors [Segal, M. S. 2006]. The latter finding supported the notion that restoration of autologous CD34⁺ cell function was a reasonable option versus substitution of healthy allogeneic cells. Because the level of TGF-B, a key factor modulating stem cell quiescence is increased in the serum of type 2 diabetic patients, we initially tested whether transient TGF-β1 inhibition in CD34⁺ cells improves reparative capacity. To inhibit TGF-β1 protein expression, the inventor treated ex vivo CD34⁺ with TGF-β1-PMOs and observed that transient inhibition of TGF-β1 resulted in substantial improvement of key in vitro functions, and more importantly, restored reparative function in vivo. It was tested herein whether the reparative function of diabetic progenitors could be enhanced through inhibition of PAI-1, the principal gene product of TGF-β1. It was determined that pre-treating CD34⁺ cells with siRNA, lentivirus shPAI-1RNA, or miR-146a resulted in a reduction in PAI-1 mRNA and protein secretion. This resulted in a beneficial response that included enhanced proliferation and migration in vitro as well as homing in vivo. Blockade of PAI-1 released the cells from G0, pushing them into G1. PAI-1 is considered as a senescence protein and removing it clearly corrected this profound cell cycle arrest observed in diabetic progenitors (Rosso, Arturo et al, 2006). It was also demonstrated herein that if PAI-1 is inhibited then cells can grow following only one day of growth factor exposure. Subsequent growth factor withdrawal did not result in cell death, but proliferation, and in the absence of growth factors, human diabetic CD34⁺ cells survived for greater than a week ex vivo. In vivo, an enhanced reparative response due not only to increased migration of the PAI-1 inhibited CD34⁺ cells, but also likely due to increased proliferation at the site of vascular injury was observed. Correction of defective homing in the diabetic CD34⁺ cells that had PAI-1 blocked was also observed herein. To better understand the mechanisms of this beneficial effect, the effect of PAI-1 blockade on its putative receptor, LRP-1, was examined. The findings showed that decreasing PAI-1 expression was associated with reduced LRP-1 surface expression. Furthermore, these cells had enhanced PI3K/AKT activation; which is supported by their improved proliferative capacity.

It was also confirmed herein that enhanced PI3K/AKT activation is responsible for increased migratory responses observed in the diabetic CD34⁺ cells under PAI-1 blockade. PI3K and subsequent Akt activation results in eNOS activation by phosphorylation at Ser1177. This results in NO generation needed for CD34⁺ cell migration [Aicher, A et al, 2003]. Healthy CD34⁺ cells were found to demonstrate robust NO release and cGMP production in response to SDF-1α. PAI-1 inhibition only slightly further increased NO release, although no changes in cGMP level. This suggests that CXCR-4 activation required for NO release in response to SDF-1 was likely near maximal in non diabetic cells before PAI-1 inhibition.

Based on the inventor's in vitro findings, PAI-1 blockade in diabetic cells will improve vascular repair by also increasing proliferative potential of these cells. Retinal and sub retinal ischemia contributes to visual impairment and blindness in diseases as diverse as retinopathy of prematurity, diabetic retinopathy and age-related macular degeneration. The I/R model mimics many aspects of the pathophysiology of retinal ischemia and leads to development of acellular capillaries, which are very similar to the vasodegenerative phase of diabetic retinopathy but appear in a markedly accelerated manner in this model. The inventor has previously shown that in this model, healthy CD34⁺/endothelial precursors reendothelialize ischemic capillaries; however, diabetic CD34⁺/endothelial precursors cells do not [Caballero, Sergio et al, 2007].

It is presented herein that inhibition of PAI-1 enhanced recruitment of diabetic and healthy CD34⁺ cells to sites of retinal injury and corrected defective repair in the diabetic CD34⁺ cells. It is shown herein that inhibition of PAI-1 in diabetic CD34⁺ cells ex vivo enhances repair following vascular damage. This finding has a profound impact on disease states associated with vascular dysfunction such as ischemic heart disease and diabetic vascular complications. While an attempt is being made to replace traditional approaches for alleviating tissue ischemia (e.g., stents, angioplasty, or vessel grafts) with cell therapy, autologous cell therapy is limited in diabetic patients because of dysfunctional cells. Inhibition of PAI-1 represents a promising therapeutic strategy for restoring vascular reparative function in diabetic CD34⁺ cells.

### Material and Methods for Examples 1-8

### Patient Selection and Characterization

Peripheral blood was collected from both type 2 and type 1 diabetic patients as well as from sex- and age-matched healthy controls. Participants gave consent to participate in this study. The study was approved by the Institutional Review Board of University of Florida. Diabetic subjects were between 18 and 65 years old and had ETDRS retinopathy score of <53. Patients having HIV, Hepatitis B or C, ongoing malignancy, current pregnancy, or history of organ transplantation were excluded from this study. Pertinent characteristics of the patients are described in Table 1.

TABLE 1

| | | |
|---|---|---|
| Patient Characteristics | Type 2 Diabetic | Type 1 Diabetic |

Healthy

| | | |
|---|---|---|
| Number | 17 | 7 |
| Age | 58.8 ± 12.86 y | 34.513 ± 9.8 y |
| Gender M/F | 12/5 | 4/3 |
| Duration of Diabetes | 14.8 ± 9.8 y | 13.7 ± 5.58 y |

Clinical

| | | |
|---|---|---|
| Smoking | 4 | 1 |
| Hypertension | 11 | 3 |
| CVD | 4 | — |
| Diabetic Retinopathy | 5 | 1 |
| Diabetic Nephropathy | — | 2 |
| Diabetic Neuropathy | 6 | 1 |

Metabolic Data

| | | |
|---|---|---|
| Glucose | | |
| HbA_{1C} | 8.9 ± 1.9 | 6.88 ± 0.29 |
| Cholesterol | | |
| Creatinine | | |

Medications

| | | |
|---|---|---|
| Oral hypoglycemic | | |
| Metformin | 7 | — |
| Thiazolidinediones | | |
| Sulfonylureas | 1 | — |
| Combinations | | |
| Aspirin | 1 | |
| Statin | 3 | 4 |
| Lipitor | 2 | |
| Angiotensin converting enzyme | 1 | |
| inhibitors/aldose reductase | | |
| inhibitors | | |

### Analysis of Plasma for PAI-1 and for TGF-β1:

Blood was collected in EDTA tubes and centrifuged at 1000 g for 15 mins to separate plasma. A 50 μl sample from each donor was analyzed by sandwich enzyme linked immune sorbent assay (ELISA) using commercially available assay kit (Quantikine, R&D Systems Inc., Minneapolis).

Isolation of Human CD34⁺ Cells from Peripheral Blood of Diabetic and Normal Donors

Blood was collected from patients using cell preparation tubes (CPT) with heparin (BD Biosciences, Franklin Lakes, N.J.) as anticoagulant. After density gradient centrifugation at room temperature in a swinging bucket rotor for 30 min at 2200 rpm, the buffy coat containing leukocytes was collected. RBC contamination was removed using ammonium chloride solution (Stem Cell Technologies, Vancouver, Ca). Mononuclear cells were enriched for CD34⁺ cells by positive selection using human CD34⁺ cell enrichment kit (Stem Cell Technologies, Vancouver, Ca). In selected studies, CD34⁺ cells maintained in culture in Stem Span median (Stem Span, Stem Cell Technology, Vancouver, Ca) supplemented with cytokine cocktails.

### Collection and Analysis of Conditioned Media

CD34⁺ EPCs (30,000 cells/well) were incubated with 100 μl stem span media (Stem Span, Stem Cell Technology, Vancouver, Ca) with Stem Span CC100 cytokine cocktail (Stem Cell Technology, Vancouver, Ca) and antibiotics for 24 hours yielding conditioned media (CM). The CM was collected for analysis of PAI-1 protein. An ELISA kit (Quantikine, R & D Systems) was used to quantify PAI-1 in the CM. The PAI-1 values were expressed as picograms per 3000 cells.

### Ex-Vivo Pre-Treatment of CD34⁺ Cells Using PMO

CD34⁺ cells isolated from normal and diabetic subjects were pretreated with 40 ng/ml of either scrambled PMO or TGF-β1-PMO overnight at 37° C. in Stem Span (Stem Cell Technologies, Vancouver, Ca) as previously described {{119 Bhatwadekar, Ashay D. 2010;}}.

### Real Time PCR

Total RNA was extracted from the cells with trizol as per manufacturer's protocol. One microgram of total RNA was transcribed using an iScript cDNA synthesis kit (Bio-Rad, Hercules, Calif.) according to manufacturer's protocol and Real Time PCR was performed using ABI Master Mix (ABI Biosystems, Foster City, Calif.). FAM labeled primers for PAI-1 was used (ABI Biosystems, Foster City, Calif.). All samples were normalized to β-actin (ABI Biosystems, Foster City, Calif.). Real Time PCR was performed on an ABI 7500 Fast PCR instrument for 40 cycles.

CD34⁺ Cell Infection with Lenti Virus

Lentivirus expressing PAI-1 shRNA and scrambled shRNA were prepared as previously described. The CD34⁺ cells were centrifuged at 300 g for 5 minutes and supernatant was removed. The cell pellet was resuspended in DMEM (high glucose), polybreen (10 μg/ml), 10% FBS to a final concentration of 5×10⁴ cells/ml. Cells were then infected with lentivirus expressing non specific shRNA or lentivirus expressing PAI-1 shRNA with a multiplicity of infection of ˜35. Cells were centrifuged at 23° C. at 150 g for 2 hours. After infection, cells were washed with PBS and cultured in Stem Span (Stem Cell Technologies, Vancouver, Ca) with/without added growth factors for the desired time period or injected into control mice, or mice undergoing injury. Uninfected cells were used as a second control.

### Cell Viability Assay

Cell viability was assessed using trypan blue exclusion and number of cells that excluded the dye was counted using a hemocytometer.

siRNA Transfection

Freshly isolated CD34⁺ cells were transfected with scrambled siRNA or PAI-1 siRNA using lipofectamine (Invitrogen) as the transfecting reagent. Opti-MEM I reduced serum medium was used as the transfection medium. Transfection was performed as per manufacturer's instructions (Invitrogen).

### Cell Migration Assay of CD34⁺ Cells by Boyden Chamber

Cell migration was performed using the modified Boyden Chamber Assay. Briefly, cells were suspended in EBM-2 media and 10,000 cells were placed per well. Wells were covered with 5 μM pore membrane coated in type 1 collagen. The assembled chamber was inverted and placed for 2 hours at 5% CO₂ to allow cell attachment to the membrane. Chambers were place right side up and 100 nM of the chemo-attractant SDF-1α was added to the top chamber and placed inside the incubator for 18 hrs. Chambers were disassembled, adhered cells were scraped from the surface and the membrane was fixed and stained. Only cells that had migrated through the membrane were counted.

### Cell Cycle Analysis

A stock solution of HØ dye (DNA intercalater) was freshly thawed and serially diluted with warm IMDM+10% FBS. Each cell sample was resuspended in 50-1000 μL, of media (either IMDM+10% FBS, or culture medium for the sample condition) and the cell suspension was added to the HØ. Cells were placed at 37° C. to incubate for 1 hr, protected from light. Twenty minutes later, the cells were removed briefly from the incubator and Pyronin Y (mRNA detector) was added. Cells were gently mixed and placed into the incubator for 40 minutes. One hour post HØ exposure, samples were pelleted, supernatant aspirated and cold blocking buffer added. After 10 minutes of incubation at 4° C., in the dark, desired surface antibodies were added and allowed to incubate for a minimum of 20 minutes. Cells were washed with FACS buffer, then re suspended in an appropriate amount of the same buffer and stored at 4° C. in the dark until FACS acquisition.

Single color compensation controls for each mouse monoclonal antibody were made using the BD™ CompBeads kit per manufacturer's instruction (BD Biosciences USA). Two aliquots of cells were stained either with HØ only or with Pyronin Y to create the nucleic acid dyes compensation controls.

### Cell Survival Assay

The cells were treated with PAI-1siRNA as described above and the cell cultures were observed and counted on day 5 and day 7. The cells were exposed to growth factors for a period of 24 hrs, after that there was a growth factor withdrawal, and then the cells were without any added growth factors for the rest of the period.

CD34⁺ Cell Transfection for the miRNA Mimic

Pre-miR miRNA precursor molecules (miR-146a mimic) were purchased from Ambion, dissolved into nuclease free water, and the resulting 50 μM stock was stored in aliquots at −80° C. CD34⁺ cells (6×10³ cells/well) were transfected with 20 nM, 40 nM, or 60 nM of precursor or negative control using Lipofectamine 2000 (Invitrogen) according to manufacturer's instructions. CD34⁺ cells transfected with miR-146a mimic were incubated for 24 hours and supernatant from cells were collected for measurement of PAI-1 secretion. Cell pellets were used for RNA isolation and Real Time PCR analysis.

### PI3 Kinase Activity Assay

Activation of PI3 Kinase by blocking PAI-1 was evaluated by measuring PI(3,4,5) P₃ synthesis in CD34⁺ cells using PI(4,5)P₂ as substrate. Briefly, cell suspension was incubated with either scrambled siRNA or PAI-1 siRNA. Following incubation the cells were lysed with lysis buffer containing 137 mM NaCl, 20 mM Tris-HCl, pH 7.4, 1 mM CaCl₂, 1 mM MgCl₂, 0.1 mM sodium orthovandate, 1% Igepal (Sigma) and 1% PMSF (Sigma) for 20 mins on ice. The lysate was collected and the protein concentration was measured by BCA Protein Assay (Pierce). Lysates were incubated with 5 ul of anti-PI3 kinase antibody (Upstate Biotechnology) at 4° C. for overnight, followed by addition of the 50% Protein A-agarose beads (Santacruz Biotechnology) addition and incubation for 2 hrs at 4° C. Immunoprecipitates were washed three times with a wash buffer consisting of 137 mM NaCl, 20 mM Tris-HCl, pH 7.4, 1 mM CaCl₂, 1 mM MgCl₂, 0.1 mM sodium orthovandate and 1% Igepal followed by washes with a wash buffer consisting of 0.1 M Tris-HCl, pH7.4; 5 mM LiCl and 0.1% Na₃VO₄ and with another buffer consisting of 10 mM Tris-HCl, pH 7.4, 150 mM Nacl, 5 mM EDTA)+0.1 mM sodium orthovandate.

Immunoprecipitated enzyme was added to the well of 96-well microplate, coated with PI(4,5)P₂. ELISA was done according to manufacturer's instruction (Echelon Biosciences, USA). The enzyme activity was expressed as amount of PI(3,4,5)P₃ produced/μg of cell protein.

Determination of cGMP Levels:

To quantify cGMP levels, an enzyme fragment complementation (EFC) technology based kit (Hithunter cGMP Assay, Fremont, Calif., USA) was used. Briefly, 10 ul of the cell volume (containing 20,000 cells) were added in one well of 384 well plate, followed by addition of 5 ul of the agonist SDF-1α. The cells were incubated with the agonist for 4 hrs. Following incubation, cGMP antibody/lysis mix and 10 ul cGMP enzyme detector reagent was added and the luminescence was measured at 1 sec interval by plate reader (Biotek).

Quantification of miRNA and mRNA Expression Level by Real Time PCR

Total RNA of CD34⁺ cells were isolated using Trizol reagent following the manufacturer's protocol. RNA concentrations were determined using NanoDrop ND-1000 spectrophotometer (NanoDrop Technology Inc, Wilmington, Del.). MiRNA analysis was done using the TaqMan MicroRNA Reverse Transcription Kit, TaqMan Universal PCR Master Mix and TaqMan MicroRNA Assay Primers for human miRNAs (Applied Biosystems, Foster City, Calif.). For mRNA analysis, iScript cDNA synthesis kit (Biorad) and Taqman mRNA assay primers for PAI-1 was used. Cycle threshold values (Ct), corresponding to the PCR cycle number at which fluorescence emission reaches a threshold above baseline emission were determined and miRNA expression values calculated using RNU6B as endogenous control following the 2-ΔΔCt method. After normalization to beta actin mRNA expression values were quantified in the same way.

### Microarray Analysis and Real Time RT-PCR

RNA from CD34⁺ cells was extracted using Trizol followed by AffyNugen amplification, and cDNA was probed to Human RSTA Affymetrix 2.0 chip using ultra low input protocol. After normalization, analysis of data was performed using one way ANOVA and changes in gene expression were further analyzed through the use of Ingenuity Pathways Analysis (Ingenuity® Systems, http://www.ingenuity.com/). Transcripts mapped in pathways analysis software were confirmed using quantitative real time RT-PCR. See the supplement for the detailed methods.

### Animal Studies

All studies were approved by the institutional animal care and use committee, and studies were conducted in accordance with The Guiding Principles in the Care and Use of Animals (NIH) as well as the ARVO Statement for the Use of Animals in Ophthalmic and Vision Research.

### Acute Vascular Injury: Ischemia Reperfusion (I/R) Injury in Eye

For this study mice that were used were 10-14 weeks, female C57/BL6J, purchased from the Jackson Laboratory. Both non-diabetic and diabetic human CD34⁺ cells were used in this study. The CD34⁺ cells were divided into three groups, untreated, cells with scrambled siRNA and cells with PAI-1 siRNA and were injected into the eye of the mouse having Ischemia/Reperfusion (I/R) Injury. The injury was done as previously described {{36 Caballero, Sergio 2007;}}.

### Mouse Model of Diabetes and Hind Limb Ischemia

The db/db mouse is a very good model for studying vascular dysfunction in type 2 diabetes. Mice that were used in this study were adult male diabetic (BKS.Cg-Dock₇^{m}+/+Lepr^{db}/J) and non-diabetic healthy heterozygotes (Dock₇^{m}+/+Lepr^{db}), 10-14 weeks old, purchased from the Jackson Laboratory. See the supplement for the detailed method.

### Bone Marrow Isolation and Enrichment for Hematopoietic Stem Cells (HSCs)

For the bone marrow isolation db/db mouse was used, and for this strain the control used was db/m. The femur and tibia was removed from each of them and was placed on ice. Both the ends of the bones were removed and the bones were flushed with ice-cold phosphate buffered saline (PBS) with a 22 gauge needle. The bone marrow was collected in a 15 ml tube and was centrifuged at 1200 rpm for 10 mins at 4° C. The supernatant was discarded and the pellet was dissolved in 2 ml PBS containing 2 mM EDTA and 10% FBS. The red blood cells (RBC) were removed by incubating the cells with 1 ml of ammonium chloride (Stem Cell Technology, Vancouver, Canada) for 15 mins on ice. The reaction was stopped by adding 10 ml of fresh buffer and the tubes were centrifuged at 1200 rpm for 10 mins. The supernatant was discarded and the cell pellet was again re-suspended in fresh buffer and was centrifuged. After this final washing, the number of cells was counted using a hemocytometer. The cells were dissolved at a concentration of 2×10⁸ cells/ml in the same buffer containing 2% rat serum and were transferred into FACS sorting tube. In that cell suspension mouse hematopoietic progenitor cell enrichment cocktail (Stem Cell Technology, Vancouver, Canada) was added and the tubes were incubated at 4° C. for 15 mins. The tubes were filled with buffer up to 2.5 ml of volume and were centrifuged for 1200 rpm for 15 mins. The supernatant was discarded and the pellet was re-suspended in fresh buffer. In that cell suspension mouse biotin selection cocktail was added and the tubes were incubated for 15 mins at 4° C. After the incubation micro-particles were added and were incubated at 4° C. for 10 mins. Then the tubes were topped off up to 2.5 ml with more media and were placed inside the magnet. After 3 mins the tubes were inverted while inside the magnet and the supernatant was poured of in another fresh tube. The new tubes containing the cells were again put inside the magnet and the step was repeated from two times. After the magnet steps the cells were counted and were then dissolved into PBS containing 1 mM EDTA and 2% FBS at a concentration of 2×1. SCA1 PE labeling reagent was mixed to it and was incubated at room temperature for 15 mins, followed by addition of PE selection cocktail and incubation for 15 mins at room temperature. After that nanoparticles were added and incubated at room temperature for 10 mins. The tubes were topped off with the media up to 2.5 ml and were placed inside magnet for 5 mins. The supernatant was poured off from the tubes by inverting the magnet, the tubes were removed from the magnet and were topped again with 2.5 ml of media, and the tubes were again placed inside magnet. The steps were repeated for twice, and the positively selected cells are ready to use. The cells enriched by this method are lin(−) ckit (+) and sca1(+).

Transfection of Mouse HSC by siRNA

The freshly isolated cells were dissolved in SS media containing mouse IL-3, IL-6, SCF (R&D Biosystems, USA) at a final concentration of 20 ng/ml, 20 ng/ml and 50 ng/ml) at a concentration of 6000 cells/100 ul. The control siRNA and the PAI-1 siRNA were purchased from Ambion. The final concentration of siRNA used was 0.05 nM. The transfection was carried out in 96 well format round bottom plate. Firstly, 1.2 ul of the respective siRNA was pipette put in each well of the plate, followed by addition of OptiMEM and lipofectamine. The reagents were incubated for 15-20 mins at RT. After the incubation, the 100 ul of media containing 6000 cells were plated on top of the transfection reagent. The plate was placed inside the incubator for 24 hrs. After 24 hrs, the cells were transferred into centrifuge tubes and washed twice with PBS. The washed cells were re-suspended in fresh PBS and were injected into the femoral artery of mouse having hind limb ischemia

### Statistical Analyses

Data are represented as Mean±SEM. All statistical analysis was done using Graph pad 3.0 (GraphPadSoftware, San Diego, Calif., USA).

### Example 9

### Treatment of Diabetic Ulcers with Treated HSCs

Generally, when the skin of an individual is torn, cut, or punctured (wounded), the body naturally reacts to regenerate dermal and epidermal tissue to close the wound. The wound regeneration process typically includes a set of complex biochemical events that take place in a closely orchestrated cascade to repair the damage. These events overlap in time, but may be categorized into different phases, namely the inflammatory, proliferative, and remodeling phases. In the inflammatory phase, bacteria and debris are phagocytized and removed, and factors are released that cause the migration and division of cells involved in the proliferative phase. In the proliferative phase, the principal steps include angiogenesis, fibroplasias, granulation tissue formation, epithelialization, and wound contraction. Angiogenesis involves the development of new capillary blood vessels for the wound area to provide oxygen and nutrients to the healing tissue. In fibroplasias and granulation tissue formation, fibroblasts grow and form a new, provisional extracellular matrix (ECM) by excreting collagen and fibronectin. In epithelialization, epithelial cells migrate across the wound bed to cover the bed. In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis.

It is known that a number of disease states hinder the normal wound healing process. For example, individuals with diabetes often experience problems with what are termed “diabetic foot ulcers.” Diabetic foot ulcers are sores or wounds, typically, on the feet that typically occur in individuals having diabetes. Oftentimes, these diabetic ulcers occur as a direct or indirect result of nerve damage in the feet of the individual as the prolonged high blood sugar and insulin levels associated with diabetes is linked with damage to the nerves in the feet. Such nerve damage in the feet, referred to as peripheral neuropathy, can cause loss of sensation as well as cause deformities of the feet. Due to the loss of sensation, individuals with peripheral neuropathy may hurt their feet by repetitive minor trauma (e.g., by prolonged walking) or a single major trauma (e.g., by scraping skin, stepping on objects, immersing feet in hot water, cutting toenails inappropriately, or wearing ill-fitting shoes), but nevertheless may not notice such injuries. A further complication of diabetes is a reduction in blood flow to the feet due to the arterial blockage or other causes, thereby severely inhibiting the body's ability to adequately provide complete the proliferative stage of wound regeneration/healing described above. As a result, once the skin of the foot is torn, cut, or punctured, the wound healing process (e.g., the proliferative phase) may be inordinately slow in repairing the wound. Further, once a serious wound develops, the risk of infection is high as the individual's body is simply unable to heal the wound. Even further, once infection starts, the infection may be very difficult to reverse, and amputation of the affected limb is common.

A number of treatments have been proposed to speed wound healing in patients having diabetic ulcers. These treatments include the use of skin grafts or “tissue equivalents.” Tissue equivalents involve the isolation of replacement skin cells that are expanded and seeded onto or into a supporting structure, such as a three-dimensional bio-resorbable matrix, or within a gel-based scaffold. Both skin grafts and tissue equivalents are notably complex and, especially in the case of reduced blood flow to the patient's feet, are often unsuccessful.

In view of the inventors' discoveries of the improved healing potential of HSCs as treated according to the teachings herein, the inventor has recognized that the treated HSCs may be utilized in the treatment of topical wounds. Thus, according to another embodiment, the invention pertains to an improved method of treating diabetic ulcers by administering treated HSCs to a patient experiencing diabetes related lesion or wound. In a more specific embodiment, there is provided a method of treating a wound in a patient including administering topically an effective amount of treated HSCs to the wound.

In accordance with yet another aspect of the present invention there is provided a method of treating a wound in a patient including administering parenterally an effective amount of treated HSCs.

In accordance with yet another aspect of the present invention there is provided a method of treating a subject having a wound. The method includes administering via topical administration a wound composition including an effective amount of treated HSCs in the vicinity of the wound, such that HSCs may migrate and adhere to the locations of the wound and/or surrounding areas. Surrounding areas would include healthy tissues contiguous to the wound.

In accordance with yet another aspect of the present invention, there is provided a method for treating a diabetic ulcer including administering to a patient in need thereof a wound composition including an effective amount of treated HSCs. The method includes administration of the HSCs so as to deliver the treated HSCs to the wound or vicinity of the wound.

In accordance with yet another aspect of the present invention there is provided a method of ameliorating the progression of a wound in a subject including administering an effective amount of treated HSCs to the wound.

The term “wound” as used in this Example refers to any break in the epithelium. The break may have been induced from a cut, abrasion, adhesion, surgical incision, thermal, chemical, or friction burn, ulcer, or pressure, or the like, as a result of an accident, incident, surgical procedure, or the like. Wound can be further defined as acute and/or chronic. Compositions of the present invention have been found to be particularly useful in the treatment of diabetic ulcers.

In accordance with an additional aspect of the present invention, there is provided a lesion treating composition. The composition includes treated hematopoietic stem cells obtained by procuring hematopoietic stem cells from a subject and treating the procured hematopoietic stem cells ex vivo by blocking activity of PAI-1 in the cells to obtain treated hematopoietic stem cells.

In accordance with another embodiment, method of treating vascular lesions in a subject in need thereof is provided. The method includes procuring umbilical cord blood hematopoietic stem cells, treating the procured hematopoietic stem cells, ex vivo, by blocking activity of PAI-1 in the cells to obtain treated hematopoietic stem cells, and administering the treated hematopoietic stem cells to the subject. In a specific embodiment, the hematopoietic stem cells are CXCR4 negative cells. In another particular embodiment, the hematopoietic stem cells are CD105 negative cells, and in another specific embodiment, the hematopoietic stem cells are CD38 negative cells.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

### Example 10

### Reduction of TGF-β1 in LTR-HSC Decreases Time to First Cell Division

Diabetics with vascular complications exhibit reduced proliferative potential of their CD34+ progenitor cells. TGF-β1 largely regulates growth of these cells as well as maintains stem cells quiescence [24]. Thus, to clarify the effect of TGF-β1 functional inhibition on HSC function, single murine FACS purified LTR-HSC were incubated with neutralizing monoclonal antibodies to TGF-β1. At day one, essentially no cell division was observed; however by day two, ˜30% of all single LTR-HSC completed at least one cell division. The addition of anti-TGF-β1 antibody increased the proportion of LTR-HSC entering their first cell division to ˜70% of all single cells. Single LTR-HSC cultured with SCF+IL-6 eventually divided; but required up to 14 days to do so. Addition of anti-TGF-β1 antibody plus SCF+IL-6 reduced this time to approximately ˜7 days.

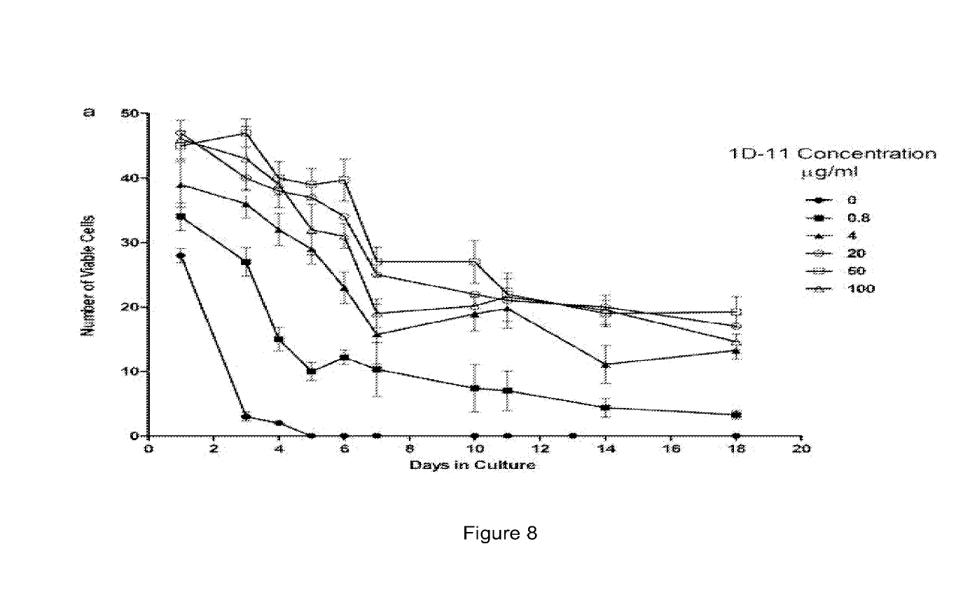

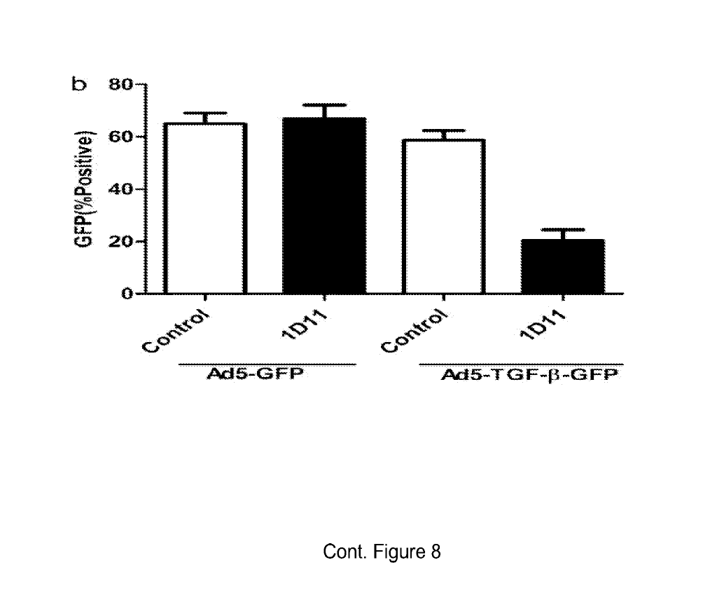

Cell division is frequently coupled to differentiation with loss of the ability to repopulate. LTR-HSC in medium alone underwent apoptosis within 3 days while cells exposed to TGF-β neutralizing antibodies at the time of plating survived for extended time periods, a response dependent on antibody concentration (FIG. 8A). Endogenous TGF-β neutralization resulted in an increased survival of LTR-HSC in the complete absence of serum or growth factors. Inhibition of TGF-β1 transcripts in LTR-HSC by PMO similarly increased LTR-HSC survival in the absence of growth factors.

Cell surface inhibition of secreted or exogenous TGF-β1 effectively down regulated the endogenous expression of TGF-β1 which is consistent with auto-regulation of transcription [25]. Levels of type II TGF-β receptors on lin−Sca-1+c-kit+ hematopoietic stem cells were then quantitated (>98% were positive). Importantly, lin−Sca-1+c-kit+ cells from TGF-β1 knockout mice (tgf-β1−/−) knockout mice expressed no type II TGF-β receptors by FACS, demonstrating that in the absence of endogenous TGF-β1 expression, the type II receptor is down regulated. Lin−Sca-1+c-kit+ cells were transduced with an adenoviral vector in which the TGF-β1 promoter drives GFP expression. These cells were treated with anti-TGF-β antibody (1D11.16) for sixteen hours which does not induce a proliferative effect. As shown in FIG. 8B, TGF-β expression was markedly reduced by 1D11.16 treatment.

Referring to FIG. 8A in particular, survival of single LTR-HSC in serum-free media in the absence of added cytokines and presence of anti-TGF-β1,2 and control and serum free or serum containing is provided. Anti-TGF-β antibody 1D11.16 increases survival of single HSC in serum-free media without added growth factors. Single LTR-HSC were directly sorted into round bottom 96 well plates containing serum or serum-free medium+/−1D11.16 (20 ug/ml), or 2G1.12 (20 ug/ml, a control MAB that binds to TGF-β2 but does not inactivate it). At the indicated day viable cells were determined. SCF, IL-3, and IL-6 were then added and 10 days later colony growth was determined (wells containing >10 cells were scored as containing a viable cell at the time point). In general, >90% of single cells formed HPP macroclones at day 10. Each combination was tested using 80 wells (single cell/well). FIG. 8B provides Anti-anti-TGF-β1,2 (1D11.16) reduces expression levels of GFP driven by a TGF-β promoter construct (Ad5-TGF-b-GFP) transduced into lin−c-kit+/Sca-1+ cells. Lin−Sca-1+HSC were transduced with pAdeno (TGF-β) GFP A/T or pAdeno GFP as control at an MOI of 100. Cultures were incubated for 16 hours at 100,000 cells/200 ul in HSC media 1D11 or IgG1K isotype control. After 24-72 hours, GFP expression was determined by FACS.

### Example 11

### Blockade of Endogenous TGF-β in LTR-HSC Significantly Increases Donor Cell Chimeras in Competitive Repopulation Transplants

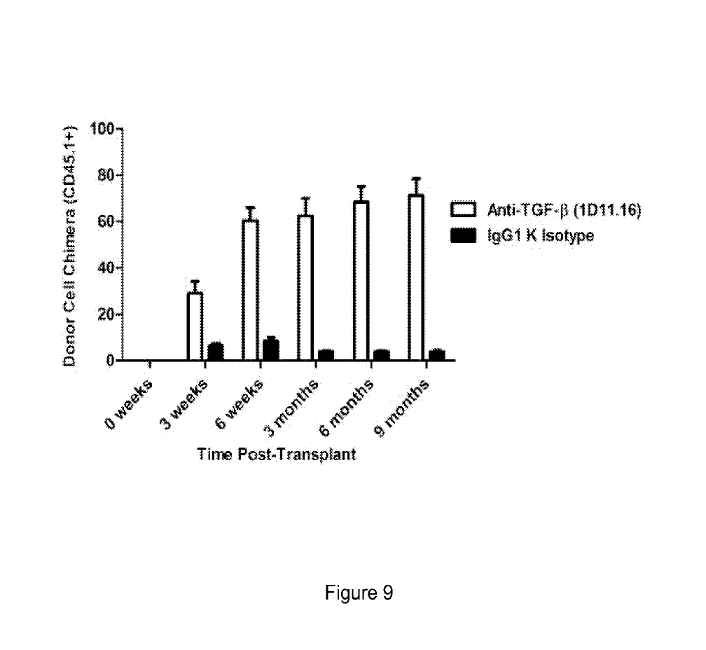

To further examine the impact of modulation of TGF-β on HSC function; a competitive congenic repopulation assay was used. Lethally irradiated CD45.2 mice (950 rads) were rescued with limiting doses (2×105 cells/mouse) of CD45.2 unfractionated (“support”) marrow and limiting numbers of CD45.1 “donor” LTR-HSCs with or without anti-TGF-β treatment. As shown in FIG. 9, donor chimerism was greater at 3 weeks in mice receiving anti-TGF-β treated LTR-HSC (30% vs.7%) and continued to climb to 6 months post transplant (70% vs. 4%).

Referring in particular to FIG. 9, LTR-HSC(CD45.2) treated for 2 hrs with anti-TGF-β1,2 antibody (1D11.16) produce a rapid and sustained donor engraftment of lethally irradiated mice (competitive repopulation using 400,000 CD45.2 BM cells/mouse). 100 LTR-HSC purified from B6SJL mice (CD45.1+) were transplanted i.v. into lethally irradiated (950 rads) congenic C57B16 (CD45.2) mice along with 400,000 unfractionated bone marrow support/competitor cells (CD45.2). The open bars (+/−S.D. n=14) show the engraftment of 100 LTR-HSC control cells (IgG1K antibody treated). The repopulation kinetics of untreated LTR-HSCs were similar to that of IgG1K antibody treated LTR-HSC (not shown). The filled bars (+/−S.D. n=19) show the engraftment of 100 LTR-HSC treated cells with ID11.16 for 2 hours just prior to transplant.

### Example 12

### TGF-β Inhibition Reduces PAI-1

PAI-1 is a major gene target of TGF-β and has been shown to be elevated in diabetes. PAI-1 is central to various pathways that regulate cellular motility (e.g., uPA, TGF-β1), proliferative (e.g., ETS, MYC, AKT), and survival/stress (e.g., JNK, caspase, NFκB, TNFR) programs [26]. Because endogenous levels of PAI-1 can be elevated in endothelial cells by exposure to high glucose, high insulin and oxidative stress, as well as in response to TGF-β, endogenous levels of PAI-1 in CD34+ cells from type 2 patients were measured herein.

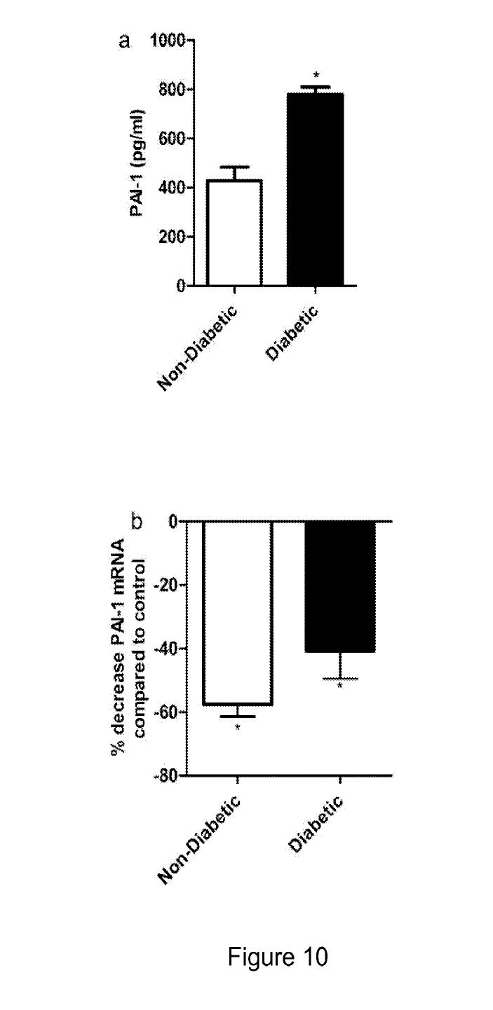

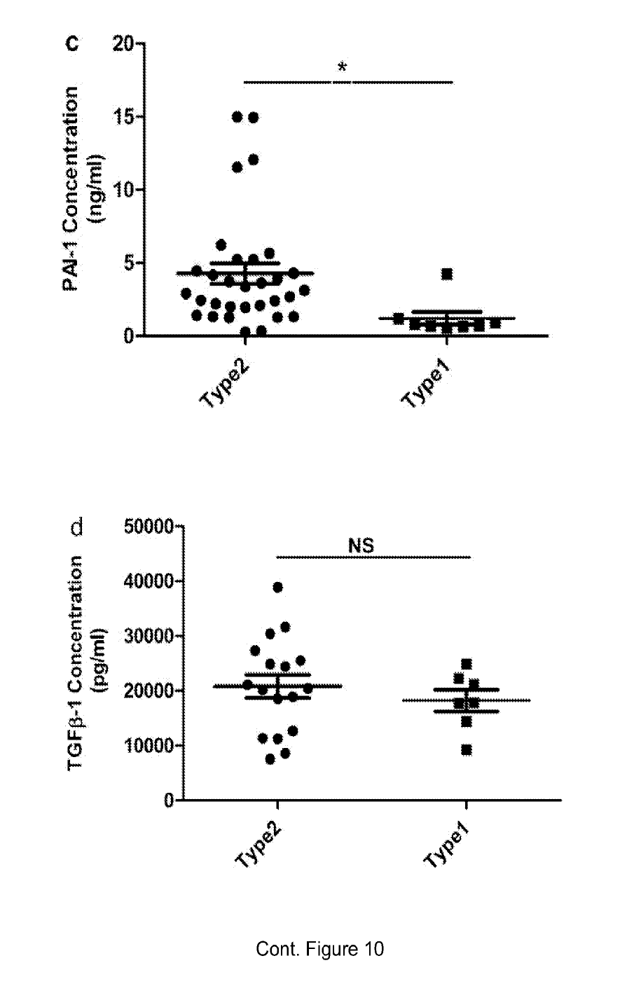

Diabetic CD34+ cells secreted significantly more PAI-1 into the CM compared to non-diabetic (p<0.05) (FIG. 10A). To assess what impact modulation of TGF-β1 would have on endogenous PAI-1 mRNA expression; CD34+ cells of diabetic or non-diabetic origin were treated with either TGF-β1-PMO or scrambled PMO prior to measurement of PAI-1 transcripts.

Decreased PAI-1 mRNA levels were evident in both diabetic and non-diabetic CD34+ cells treated with TGF-β1 PMO compared to cells transfected with scrambled PMO, (p<0.001 diabetic and p=0.05 non-diabetic) (FIG. 10B). As these progenitors not only produce TGF-β and PAI-1 but are exposed to plasma levels of these factors, we next quantified plasma PAI-1 and TGF-β1 in these same type 2 patients for comparison to a group of type 1 subjects. (FIGS. 10 C, D). Plasma PAI-1 levels were higher in type 2 diabetics compared to type 1 diabetics (n=31 for type 2, n=8 for type 1, p=0.03 FIG. 10C) while TGF-β1 levels were similar in both groups (n=17 for type 2, n=7 for type 1 FIG. 10D). A positive correlation was evident (coefficient of correlation 0.44), however, when plasma TGF-β1 levels were compared with PAI-1 in type 2 diabetics (FIG. 10E), In contrast, a negative correlation was found between PAI-1 and TGF-β1 in type 1 diabetes (FIG. 10F; correlation coefficient −0.39).

Because protected diabetic patients exhibited lower PAI-1 levels, the inventors determined that inhibition of PAI-1 may have a beneficial effect on CD34+ cell function. Three separate approaches were used to reduce PAI-1 in CD34+ cells, PAI-1 siRNA, lentivirus expressing PAI-1 shRNA and over expressing miR-146a mimic. The efficiency of the knockdown effects are shown in FIG. 10. Referring particularly to FIG. 10, TGF-β1 mediates its action through PAI-1 in both diabetic and non-diabetic CD34+ cells. FIG. 10A shows PAI-1 concentration in the conditioned media of diabetic and non-diabetic CD34+ cells. There was a significant increase in the secreted level of PAI-1 in the conditioned media obtained from the diabetic CD34+ cells compared to non-diabetic (p<0.05) (mean±SEM; n=3). In FIG. 10B, the effect of control PMO and TGF-β1-PMO on PAI-1 gene expression in CD34+ cells was assessed. Both diabetic and non-diabetic CD34+ cells were pretreated overnight with either scrambled PMO or TGF-β1-PMO (40 ng/ml). PAI-1 mRNA transcripts were quantified by RT-PCR and was normalized to β-actin level. Values in cells treated with scrambled PMO were set at 1.0. p<0.001 (for non-diabetic compared to scrambled PMO treated cells); p=0.05 (for diabetic compared to scrambled PMO treated cells); n=10 for diabetic and n=3 for control. FIGS. 10 C-D show plasma concentrations of PAI-1 and TGF-β1 in type 1 and type 2 diabetics. There was a significant increase in the plasma 29 concentration of PAI-1 in type 2 diabetics compared to type 1 (n=31 for type 2; n=7 for type 1), although the concentration of TGF-β1 was similar in both groups. FIG. 10E shows that a positive correlation was found between the plasma level of PAI-1 (ng/ml) and plasma level of TGF-β1 (pg/ml) in type 2 diabetes with r=0.44. FIG. 10F demonstrates that a negative correlation was found between the plasma level of PAI-1 (ng/ml) and plasma level of TGF-β1 in type 1 diabetes with r=−0.39. Each dot represents one patient sample.

### Example 13

### PAI-1 Blockade Eliminates the Inhibitory Effect of TGF-β1 on CD34+ Cells and Increased Proliferation of Healthy and Diabetic CD34+ Cells

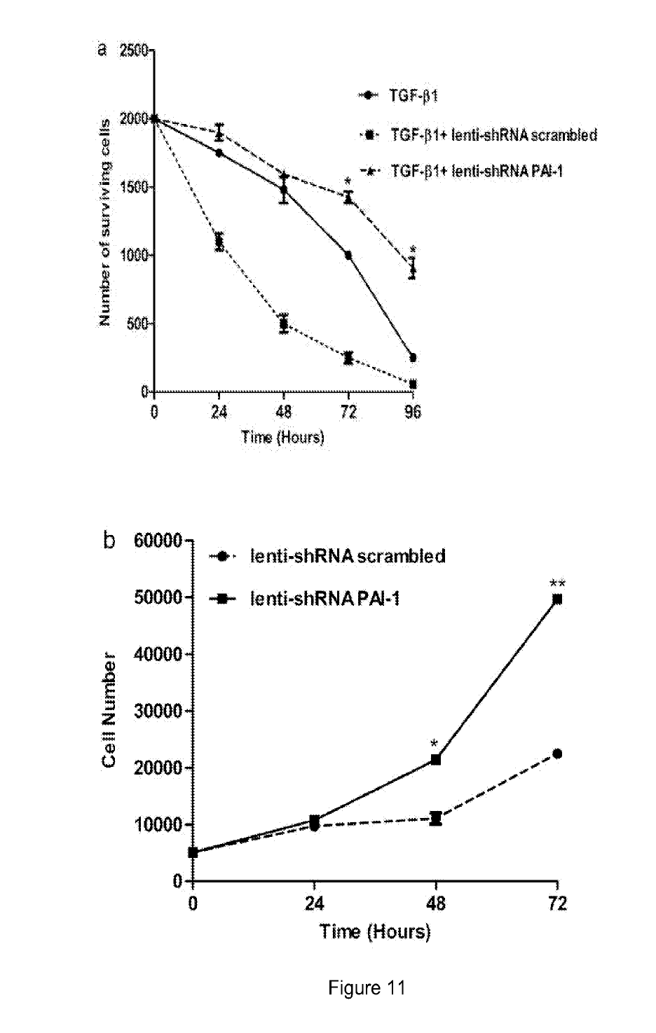

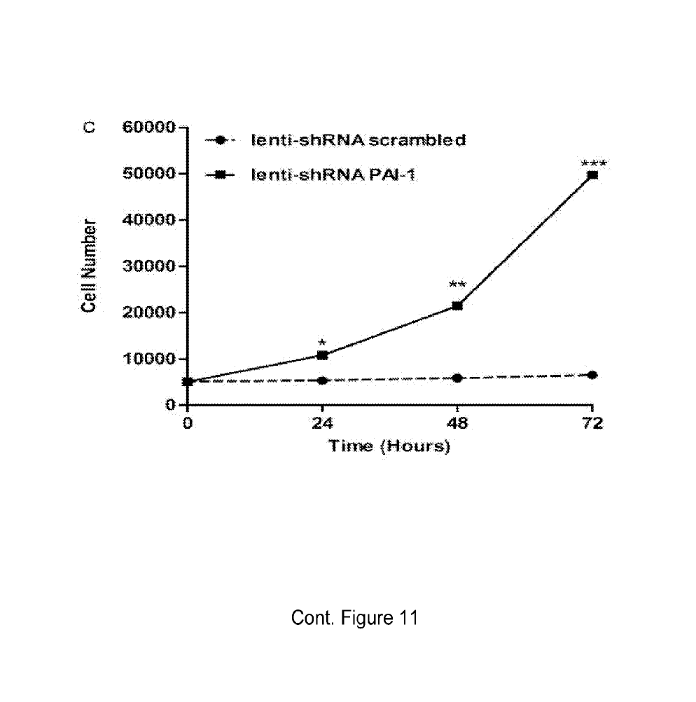

CD34+ cells express low-density lipoprotein receptor-related protein 1 (LRP-1), the putative receptor for PAI-1 [27], supporting that PAI-1 may mediate both paracrine and autocrine effects on CD34+ cells. To distinguish the effects of TGF-β1 from PAI-1, CD34+ cells were exposed to both TGF-β (1 ng/ml) and either lentivirus expressing PAI-1 shRNA or scrambled shRNA and determined cell viability over 72 hrs. As shown in FIG. 11A, cells exposed to TGF-β without addition of growth factors had a marked decrease in cell number that became more pronounced over time (solid line with closed circle). In cells exposed to lentivirus expressing scrambled shRNA and TGF-β1, a similar decreased over time (dotted line with closed square) was observed but more pronounced likely due to the potential toxicity of the lentivirus on these cells [28]. In contrast, when the cells were infected with PAI-1 shRNA lentiviruses and TGF-β1, there was significant increase in the number of surviving cells even in the absence of growth factors (p<0.001 for TGF-β1+PAI-1shRNA compared to TGF-β1+ scrambled shRNA) (dotted line with closed triangle). These results provide that TGF-β1 mediates the inhibitory effects of PAI-1 on CD34+ cells.

In the presence of growth factors, inhibition of PAI-1 promoted cell proliferation both in diabetic and nondiabetic CD34+ cells (FIG. 11B, C). Non-diabetic CD34+ cells (FIG. 11B) proliferated following PAI-1 blockade (solid line) to a greater degree than cells treated with lenti shRNA control (dotted line). In contrast, diabetic CD34+ cells did not proliferate (dotted line) even in the presence of growth factors; however, blocking PAI-1 remarkably increased the proliferation rate of the diabetic cells (solid line) to the level of non-diabetic cells (FIG. 11C).

An important issue for cell therapy is the apparent need to ex vivo expand CD34+ cells, in the absence of differentiation, prior to their re-introduction into patients. To determine whether PAI-1 blockade could mediate such an effect, the number of cells in G0 and in G1 were assessed at days 5 and 7 at baseline conditions and following PAI-1 siRNA treatment. Following PAI-1 siRNA treatment, fewer cells were in G0 suggesting that reducing PAI-1 facilitated the transition of cells through the cell cycle (data not shown). Moreover, to minimize the exposure of progenitors to growth factors to reduce risk of differentiation and to allow for expansion, CD34+ cells were treated with PAI-1siRNA in the presence of growth factors for only 24 hrs and then the growth factors were removed. Compared to control siRNA treated cells, inhibition of PAI-1 allowed a greater number of cells to survive in the absence of growth factors over 6 days (78.5% increase compared to control siRNA).

In regard to FIG. 11, it was determined that PAI-1 blockade eliminates the inhibitory effect of TGF-β1 on CD34+ cells and increased proliferation of healthy and diabetic CD34+ cells following 24 hr exposure. In FIG. 11(a), non-diabetic CD34+ cells were infected with lentivirus expressing PAI-1 shRNA or scrambled shRNA. After 24 hours the cells were treated with recombinant TGF-β1 (1 ng/ml) and at every 24 hrs the number of viable cells was counted using trypan blue up to 72 hrs. The solid line represents control cells, top broken line represents cells infected with PAI-1shRNA lentiviruses and bottom broken line represents lentivirus expressing scrambled shRNA. p<0.001 for scrambled shRNA+TGF-β1 vs. PAI-1 shRNA+TGF-β1. Each data point represents mean±SEM for 3 separate experiments in duplicate. In FIG. 11(b) Non-diabetic and FIG. 11(c) diabetic CD34+ cells were infected with either lentivirus expressing PAI-1 shRNA (solid line) or lentivirus expressing scrambled shRNA (broken line) for 2 hours and then cultured with added growth factors (cytokine cocktail) for up to 72 hours. After every 24 hr period, the number of viable cells were counted using trypan blue. Each data point represents mean±SEM for 3 separate experiments in duplicate.

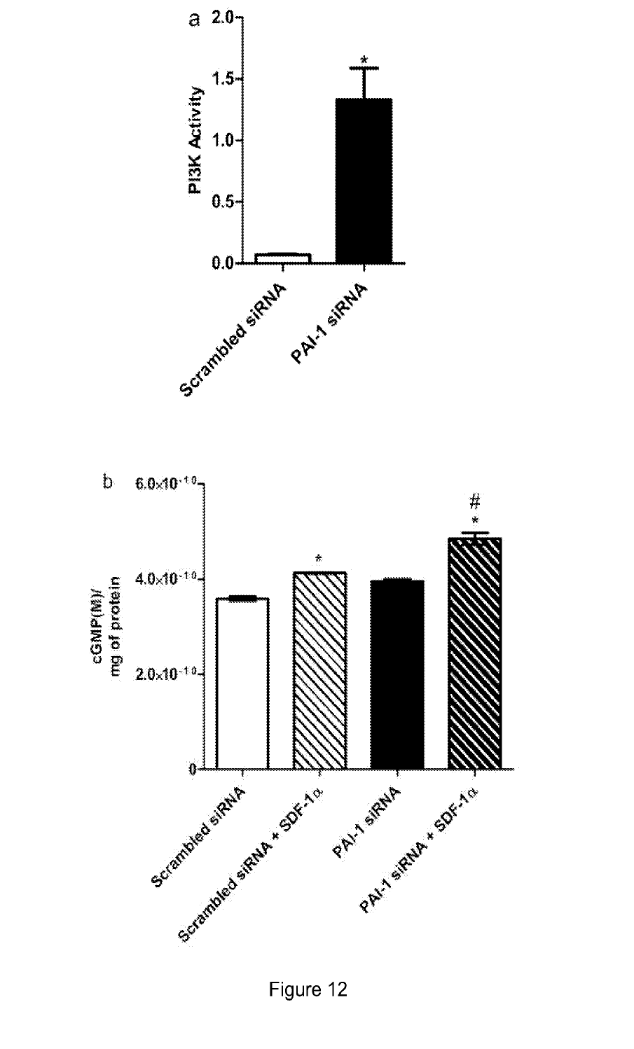

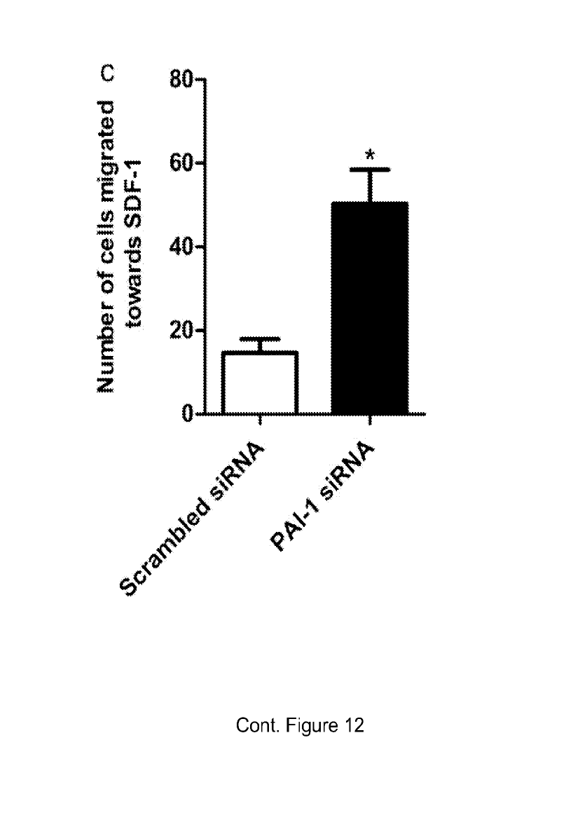

In addition, studies on mouse embryo fibroblasts (also human fibroblasts) indicated that PAI-1 knockdown leads to cell cycle progression by increasing phosphatidylinositol 3-kinase (PI(3)K) signaling [17], we asked whether this also occurred in CD34+ cells in which PAI-1 was reduced. The effect of inhibition of PAI-1 on PI3K activity in CD34+ cells was tested using the conversion of PI(3,4,5)P3 to PI(4,5)P2. In CD34+ cells, blocking PAI-1 stimulated PI(3)K activity significantly compared to scrambled siRNA treatment (p<0.05) (FIG. 12A).

### Example 14

### PAI-1 Inhibition Improved Migration of Diabetic CD34+ Cells

Bioavailable NO is important for the homing and migration of progenitor cells [29]. In diabetes, typically CD34+ cells demonstrate reduced NO bioavailability [30]. As PI3 (K)-AKT signaling is related to eNOS expression, it was necessary to determine whether inhibition of PAI-1 was associated with increased cGMP production. In diabetic CD34+ cells, inhibition of PAI-1 increased cGMP production under both basal and SDF-1 (100 nM/L) stimulation by 10% and 17% respectively (FIG. 12B). Moreover, PAI-1 inhibition improved the migratory response of CD34+ diabetic cells to SDF-1α compared to control scrambled siRNA treatment (FIG. 12C), suggesting that reducing PAI-1 in the diabetic cells corrected their migratory dysfunction in vitro.

In particular, with reference to FIG. 12, it can be seen that PAI-1 inhibition increases PI (3) K activity, CGMP production and migration of the diabetic CD34+ cells. FIG. 12A shows PI3 kinase activity was measured in nondiabetic CD34+ cells by measuring the amount of PI(3,4,5) P3 produced from PI(4,5) P2 following PAI-1 inhibition. The amount of product produced was measured by ELISA. The bar graph is the representative of 3 separate experiments. FIG. 12B provides a representation of cGMP production after PAI-1 inhibition was measured by chemiluminescence assay. FIG. 12C provides a Boyden chamber assay showing migration of diabetic CD34+ cells to 100 nM of SDF-1α. Freshly isolated cells were exposed to either PAI-1 siRNA (5 nM) or scrambled siRNA and were then allowed to migrate towards SDF-la (100 nM) for 18 hrs. Numbers of migrated cells were counted. The graph shows the number of cells that migrated cells after being pre-exposed to either scrambled siRNA (black bar) or PAI-1 siRNA (white bar).

Several of the novel discoveries disclosed herein, but never heretofore discovered include but are not limited to the following: first, patients protected from development of microvascular complications have lower TGF-β/PAI-1 transcript levels in their CD34+ cells. Secondly, reduction of TGF-β in LTR-HSC prior to transplant stimulates cell division and homing in vivo. Thirdly, rescue of progenitor cell function by endogenous TGF-β inhibition appears to be largely mediated by PAI-1 reduction which corrects diabetic CD34+ cell function in vitro.

The examples and methods and discoveries by the inventors disclosed herein emphasize the central role of TGF-β/PAI-1 system in the pathogenesis of diabetic vascular pathology. Consistently, anti-TGF-β strategies improved survival, accelerated engraftment and generated durable long-term donor engraftment compared to control treated cells. The mechanism(s) mediating the profound effects that were observed both on HSC survival in the absence of growth factors and the rapid and enhanced engraftment in irradiated recipients is likely multifactorial. The inventors showed that TGF-β inhibition was reversible regulator of LTR-HSC quiescence [14]. The role of cell cycle position on HSC bone marrow engraftment has been studied extensively. Both murine and human HSC engraft with greater efficiency at the G0/G1 phase of the cell cycle, in contrast to the low engraftment of HSC observed in the G2/S/M phase [31-34]. Thus it is not surprising, that TGF-β inhibition promoted engraftment. Studies show that blocking TGF-β mediated Smad signaling by over expressing the inhibitory Smad 7[13] increased HSC self renewal. Thus the inventors identify that blocking Smad signaling may have a cascade effect on HSC self-renewal and homing. TGF-β expression in HSC was down regulated by using either TGF-β neutralizing antibodies or PMO; however this down regulation by antibody is dependent on cell surface TGF-β receptor expression and signaling which appears to vary significantly more in human CD34+ cells than murine LTR-HSC (Ruscetti, unpublished). These murine studies show marked improvements in HSC transplantation efficiency in experimental animal models and suggests that this approach can be clinically useful in settings of limited donor HSC. HSC transplantation efficiency is dependent on migration (homing of transplanted HSC cells back to bone marrow) and HSC proliferation at microenvironmental sites and is similar to concepts of therapeutic revascularization requiring progenitor cells homing to areas of injury in order to provide paracrine support to the traumatized tissue and vascular network.

Autologous CD34+ stem cells holds promise to prevent tissue damage and restore blood flow in diabetics who are not ideal candidates for standard revascularization procedures due to diffuse vascular disease or failed previous revascularization. This autologous approach, however, is limited due to endothelial progenitor cell dysfunction [5, 9, 35, 36]. Indeed, endothelial progenitors isolated from diabetic patients demonstrate reduced proliferation, migration, and differentiation into endothelial cells [7, 37]. Interestingly the CD34+ cells derived from protected patients expressed higher levels of uPA. uPA, much like NO, is needed to promote cell migration [27], which is a major function of CD34+ cells as these cells need to home to areas of injury to facilitate repair. Consistent with this finding, CD34+ cells isolated from diabetic that have vascular complications show reduced NO bioavailability which is associated with decreased migration that can be restored through exposure to NO donors [7]. The later finding supported the notion that restoration of autologous CD34+ cell function was a reasonable option versus substitution of healthy allogeneic cells.

Treatment of CD34+ cells ex vivo with TGF-β1-PMOs to transiently inhibit TGF-β1 resulted in substantial improvement of key in vitro functions and more importantly restored reparative function in vivo [16]. Since PAI-1 is a prominent member of the TGF-β1-response gene set and functions to negatively regulate cell growth [17], it was important to determine if reparative function of diabetic progenitors could be enhanced through inhibition of PAI-1. It is identified herein that PAI-1 provides a more efficacious and potentially safer target, as PAI-1 has a narrower range of effects than TGF-β1. Pre-treatment of CD34+ cells with PAI-1 siRNA, shPAI-1 lentiviruses, or miR-146a reduced PAI-1 mRNA and protein levels resulting in enhanced proliferation and migration in vitro as well as homing in vivo. PAI-1 inhibition up-regulated G0 exit and re-entry into the pre-cycling G1 state, reversing the profound cell cycle arrest observed in diabetic progenitors [6]. It was also shown herein that if PAI-1 is inhibited, cells grow faster following only one day of growth factor exposure. Subsequent growth factor withdrawal did not result in cell death, but proliferation, and in the absence of growth factors, and human diabetic CD34+ cells survived for greater than a week ex vivo. PAI-1 inhibition in CD34+ cells was also associated with increased PI3K activity, reflective of both their improved proliferative and migratory response. While the mechanisms remain unclear, PI3K activation and subsequent Akt pathway engagement results in eNOS activation by phosphorylation at Ser1177 and leads to NO generation needed for effective cell migration. [38].

Consequently, the inventors have discovered that inhibition of the TGF-β-PAI-1 axis in diabetic CD34+ cells ex vivo enhances their function. Modulation of this pathway will provide a profound impact on disease states associated with vascular dysfunction such as ischemic heart disease and diabetic vascular complications. While an attempt is being made to replace traditional approaches for alleviating tissue ischemia (e.g., stents, angioplasty, or vessel grafts) with cell therapy, autologous cell therapy is limited in diabetic patients because of dysfunctional cells. Inhibition of TGF-β or PAI=1 (or both) may represent a promising therapeutic strategy for restoring vascular reparative function in diabetic CD34+ cells.

### Materials and Methods for Examples 10-14

### Growth Factors and Antibodies

For the murine studies, purified, recombinant growth factors were generously provided by and used as follows: rat SCF (50 ng/ml) from Dr. Krisztina Zsebo, (Amgen Inc., Thousand Oaks Calif.); murine IL-3 (10 ng/ml) from Dr. Andrew Hapel, (Australian National University); human IL-6 (10 ng/ml) from Dr. Douglas Williams (Immunex Corp., Seattle Wash.); anti-TGFβ antibodies 1D11.16, 2G1.12 and 2C7.14 from Jim Dasch (Celltrix Corp, Santa Cruz, Calif.), 2G7 from Mike Palladino (Genentech Corp, San Francisco, Calif.) and Fab2′ fragments of 1D11.16 provided by Bruce Blazar [39]. IgG1K isotype control antibodies were purchased from R&D systems (Minneapolis Minn.).

### Patient Selection and Characterization:

Peripheral blood was collected from both type 1 and type 2 diabetic patients as well as from sex- and age-matched healthy controls. This study was conducted under Institutional Review Board of University of Florida (IRB) approval # IRB 570-2008. Participants gave written informed consent to participate in this study and Declaration of Helsinki protocols were followed. Patients having HIV, Hepatitis B or C, ongoing malignancy, current pregnancy or history of organ transplantation were excluded from this study. Pertinent characteristics of the patients are described in Table S3 of the supplement document. A separate pool of patients was used in this study, were protected from vascular complications although having long-standing poorly controlled diabetes. The details are in Table Si.

### Microarray Analysis and Real Time RT-PCR

RNA from CD34+ cells was extracted using Trizol followed by AffyNugen amplification and cDNA hybridized to Human RSTA Affymetrix 2.0 chips using ultra low input protocol. After normalization, analysis of data was performed using one way ANOVA and changes in gene expression were further analyzed through the use of Ingenuity Pathways Analysis (Ingenuity® Systems, http://www.ingenuity.com/). Transcripts mapped in pathways analysis software were confirmed using quantitative real time RT-PCR.

### Culture Conditions

Single and multiple sorted cells were cultured in 96-well U-bottomed plates (Corning) in IMDM medium (Gibco BRL, Grand Island N.Y.) with 10% horse serum (HS, Gibco), 10 fetal bovine serum (FBS, Gibco), 2×10−5 M 2-mercaptoethanol (2-ME, Sigma), 10−7 M hydrocortisone (HC, Sigma) and antibiotics (penicillin/streptomycin, Gibco) (HSC media) supplemented with cytokines. Details are in supplement.

### Animals

Three- to six-month-old male congenic B6SJL CD45.1 and C57BL/6J CD45.2 mice were purchased from Jackson Laboratories (Bar Harbor Me.) and housed at Seattle Biomedical Research Institute, Seattle, Wash. and used within two weeks for transplant studies.

### Enrichment for LTR- and STR-HSC: Pre Fractionation of Bone Marrow:

Mice were sacrificed, femurs and tibias were removed aseptically, and marrow was harvested by flushing with phosphate-buffered saline containing 2% fetal bovine serum (PBS/2% FBS). Detailed methods are included in the supplement.

### Enrichment for LTR- and STR-HSC: Fluorescence Activated Cell Sorting:

The pre-fractionated cells were analyzed and sorted on a FACStar Plus flow cytometer (Becton Dickinson, San Jose Calif.) equipped with dual argon lasers, and an automated cell delivery unit (ACDU). Cells were kept chilled at 4° C. with a recirculation water bath. Monochromatic light at 351-364 nm and 488 nm was used for Hö and Rh excitations, respectively. Forward light scatter was detected using 488 bp10 and ND 1.0 filters. Hö emission was detected using a 515 long pass filter in order to maximize signals from HSC. Rh emission was detected using a 530 bandpass20 filter, PE emission using a 575 bandpass 20 filter, and PI emission using a 610 long pass filter. Cells were gated as follows: first, forward light scatter and PI fluorescence were analyzed, and viable cells (PI negative) were selected. Cells in these gates were further refined by selecting specific percentages from the Rh fluorescence histogram: the lowest 10% (defined as Rh low) and the middle 40% of the peak (defined as Rh high)₁. Then Rh low and Rh high cells were analyzed for Ho fluorescence and c-kit receptor. Cells that simultaneously demonstrated low Ho fluorescence and expressed c-kit receptor were sorted as individual cells into 96-well plates or collected in bulk. These two sorted fractions were defined as: lin−Hölowckit+Rhlow and lin−Hölowc-kit+Rhhigh, respectively, henceforth designated LTR-HSC and STRHSC.

### Long Term Repopulation Assay:

Competitive or direct transplantation was used to measure repopulation capacity or short-term survival respectively of sorted cell populations. From one to 100 LTR-HSC were directly sorted into U-bottomed 96-well plate, centrifuged at 400 g and cell numbers were directly verified with an inverted microscope using bright field at 200× magnification. Wells usually contained the expected number of cells or less, and wells with the desired cell number were marked and used for transplant. The competitive repopulation assay was performed using CD45.1/45.2 congenic mice. Recipient animals (C57BL/6J CD45.2) were exposed to a single dose 950 cGy total body irradiation and 2−4×10⁵ unfractionated (CD45.2) bone marrow cells were added to wells containing (B6SJL CD45.1) LTR-HSC donor cells and injected into the tail vein of the recipient.

Three weeks to 12 months after transplantation the proportion of donor-derived (CD45.1) nucleated leukocytes in the recipient's peripheral blood w were quantitated by FACS analysis. Peripheral blood was obtained by capillary puncture of the orbital venous plexus and 100 μl were transferred into 1 ml PBS/2% FBS, centrifuged for five minutes at ×400 g, resuspended in 100 ul of PBS/2% FBS, and red blood cells were lysed with 1 ml of NH4Cl lysis buffer for 10 minutes at 37° C. Then 2 ml of PBS/2% FBS was added; cells were centrifuged for 10 minutes at ×400 g and washed twice with PBS/2% FBS. The nucleated cells were divided into two fractions and stained with fluorochrome-conjugated monoclonal antibodies specific against either CD45.1 antigen (A20 clone) or CD45.2 antigen (104 clone, PharMingen). After staining, cells were analyzed on an Epics Profile II (Coulter Electronics, Hialeah, Fla.). Red cell contamination was eliminated by analyzing only CD45.1 and CD45.2 positive cells. Non-specific binding of anti CD45.1 antibody was determined by control binding to CD45.2 leukocytes. The frequency of long-term repopulating units was estimated by using the maximum-likelihood model that requires limiting dilution cell transplants of the test cells as described by Taswell [40].

Isolation of Human CD34+ Cells from Peripheral Blood of Diabetic and Normal Donors:

Blood was collected from patients using cell preparation tubes (CPT) with heparin (BD Biosciences, San Jose, Calif.). After density gradient centrifugation, at room temperature in a swinging bucket rotor for 30 min at 2200 rpm, the buffy coat containing leukocytes was collected. RBC contamination was removed using ammonium chloride solution (Stem Cell Technologies, Vancouver, Canada). Mononuclear cells were enriched for CD34+ cells by positive selection using human CD34+ cell enrichment kit (Stem Cell Technologies). In selected studies, CD34+ cells were maintained in culture in Stem Span median (Stem Span, Stem Cell Technology) supplemented with cytokine cocktails (Stem Cell Technology).

### Collection and Analysis of Conditioned Media:

CD34+ EPCs (30,000 cells/well) were incubated with 100 μl stem span media (Stem Span, Stem Cell Technology, Vancouver, Ca) with Stem Span CC100 cytokine cocktail (Stem Cell Technology, Vancouver, Ca) and antibiotics for 24 hrs, yielding conditioned media (CM). The CM was collected for analysis of PAI-1 protein. An ELISA kit (Quantikine, R&D Systems) was used to quantify PAI-1 in the CM. The PAI-1 values were expressed as pg per 30,000 cells.

### Ex-Vivo Pre-Treatment of CD34+ Cells Using Antisense Phosphorodiamidate Morpholino Oligomers (PMO) to TGF-β1

CD34+ cells isolated from normal and diabetic subjects were pretreated with 40 m/ml of either scrambled PMO or TGF-β1-PMO overnight at 37° C. in Stem Span (Stem Cell Technologies, Vancouver, Canada) as previously described [16].

### Real Time PCR

Total RNA was extracted from the cells with trizol as per manufacturer's protocol. 1 μg of total RNA was transcribed using an iScript cDNA synthesis kit (Bio-Rad, Hercules, Calif.) according to manufacturer's protocol and Real Time PCR was performed using ABI Master Mix (ABI Biosystems, Foster City, Calif.). FAM labeled primers for PAI-1 was used (ABI Biosystems, Foster City, Calif.). All samples were normalized to β-actin (ABI Biosystems, Foster City, Calif.). Real Time PCR was performed on an ABI 7500 Fast PCR instrument for 40 cycles. Primer details are in Table S4.

### Analysis of Plasma PAI-1 and TGF-β1:

Blood was collected in EDTA tubes and centrifuged at 1000 g for 15 mins to separate plasma. A 50 μl sample from each donor was analyzed by sandwich enzyme linked immune sorbent assay (ELISA) using commercially available assay kit (Quantikine, R&D Systems Inc., Minneapolis).

CD34+ Cell Infection with Lentivirus

Lentivirus expressing PAI-1 shRNA and scrambled shRNA were prepared as described (R M Klein and P J Higgins, in preparation). The CD34+ cells were centrifuged at 300 g for 5 mins and supernatant was removed. The cell pellet was resuspended in DMEM (high glucose), polybreen (10 μg/ml), 10% FBS to a final concentration of 5×10⁴ cells/ml. Cells were then infected with lentivirus expressing non specific shRNA or lentivirus expressing PAI-1 shRNA with a multiplicity of infection of ˜35. Cells were centrifuged at 23° C. at 150 g for 2 hours. After infection, cells were washed with PBS and cultured in Stem Span (Stem Cell Technologies, Vancouver, Canada) with/without added growth factors for the desired time period. Uninfected cells were used as a second control.

### Cell Viability Assay

Cell viability was assessed using either trypan blue exclusion and number of cells that excluded the dye was counted using a hemocytometer or using propidium iodide exclusion as detected using an LSRII flow cytometer analyser.

### Cell Cycle Analysis

A stock solution of HØ dye (DNA intercalater) was freshly thawed and serially diluted with warm IMDM+10% FBS. Each cell sample was resuspended in 50-100 μL of media (either IMDM+10% FBS, or culture medium for the sample condition) and the cell suspension was added to the HØ. Cells were placed at 37° C. to incubate for 1 hr, protected from light. Twenty mins later, the cells were removed briefly from the incubator and Pyronin Y (mRNA detector) was added. Cells were gently mixed and placed into the incubator for 40 min. One hour post HØ exposure, samples were pelleted, supernatant aspirated and cold blocking buffer added. After 10 mins of incubation at 4° C., in the dark, desired surface antibodies were added and allowed to incubate for a minimum of 20 min. Cells were washed with FACS buffer, then re suspended in an appropriate amount of the same buffer and stored at 4° C., in the dark, until FACS acquisition. Single color compensation controls for each mouse monoclonal antibody were made using the BD™ CompBeads kit as per manufacturer's instruction (BD Biosciences, San Jose, Calif.). Two aliquots of cells were stained either with HØ only or with Pyronin Y to create the nucleic acid dyes compensation controls.

siRNA Transfection

Freshly isolated CD34+ cells were transfected with scrambled siRNA or PAI-1 siRNA using lipofectamine (Invitrogen, Grand Island, N.Y.) as the transfecting reagent. Opti-MEM I reduced serum medium was used as the transfection medium. Transfection was performed as per manufacturer's instructions (Invitrogen, Grand Island, N.Y.).

### Cell Migration Assay of CD34+ Cells by Boyden Chamber Assay

Cell migration was performed using the modified Boyden Chamber Assay. Briefly, cells were suspended in EBM-2 media and 10,000 cells were placed per well. Wells were covered with 5 μM pore membrane coated in type 1 collagen. The assembled chamber was inverted and placed for 2 hours at 5% CO2 to allow cell attachment to the membrane. Chambers were place right side up and 100 nM of the chemo-attractant SDF-1α was added to the top chamber and placed inside the incubator for 18 hrs. Chambers were disassembled, adhered cells were scraped from the surface and the membrane was fixed and stained. Only cells that had migrated through the membrane were counted.

### PI3 Kinase Activity Assay

Activation of PI3 Kinase by blocking PAI-1 was evaluated by measuring PI(3,4,5) P3 synthesis in CD34+ cells using PI(4,5)P2 as substrate. Briefly, cell suspension was incubated with either scrambled siRNA or PAI-1 siRNA. Following incubation, the cells were lysed with lysis buffer. The lysate was collected and the protein concentration was measured by BCA Protein Assay (Thermo Scientific, Rockford, Ill.). Lysates were incubated with anti-PI3 kinase antibody (Upstate Biotechnology, Billerica, Mass.)) at 4° C. for overnight, followed by addition of the 50% Protein A-agarose beads (Santacruz Biotechnology, Santa Cruz, Calif.). Immunoprecipitates were washed with a wash buffer and immunoprecipitated enzyme was added to the wells of 96-well microplate, coated with PI(4,5)P2. ELISA was performed according to manufacturer's instruction (Echelon Biosciences, Salt Lake City, Utah). The enzyme activity was expressed as amount of PI(3,4,5)P3 produced/μg of cell protein.

Measurement of cGMP Production

The cGMP production in response to SDF-1α (100 nM/L) was measured by HitHunter cGMP assay kit (DiscoverRx Corporation, Fremont, Calif.) as per manufacturer's instruction. Briefly, 20,000 cells were used per treatment. The cells were treated with SDF-1α for 4 hrs and the cGMP production was compared between un-stimulated and stimulated cells. The luminescence was measured by a plate reader (Biotek Instruments, Winooski, Vt.).

### Cell Survival Assay

The cells were treated with PAI-1siRNA as described above and the cell cultures were observed and counted on day 5 and day 7. The cells were exposed to growth factors for a period of 24 hrs, after that there was growth factor withdrawal, and then the cells were without any added growth factors for the remainder of the culture period.

### Statistics

Regression models were used for time course studies with tests for differences between groups over time and group by time interaction. Multivariate techniques, assessing vectors of TGF-β and PAI-1 levels were used. Modeling methods were used to examine flow cytometry parameters between groups and over time. Tests were conducted at a 0.05 level of significance; multiple comparison procedures were used to identify specific differences.

### REFERENCES

1. Fadini, G P, et al. (2006). Circulating CD34+ cells, metabolic syndrome, and cardiovascular risk. Eur Heart J 27: 2247-2255.
2. Fadini, G P, et al. (2006). Number and function of endothelial progenitor cells as a marker of severity for diabetic vasculopathy. Arterioscler Thromb Vasc Biol 26: 2140-2146.
3. Fadini, G P, et al. (2010). Time course and mechanisms of circulating progenitor cell reduction in the natural history of type 2 diabetes. Diabetes Care 33: 1097-1102.
4. Chen, Y H, et al. (2007). High glucose impairs early and late endothelial progenitor cells by modifying nitric oxide-related but not oxidative stressmediated mechanisms. Diabetes 56: 1559-1568.
5. Caballero, S, et al. (2007). Ischemic vascular damage can be repaired by healthy, but not diabetic, endothelial progenitor cells. Diabetes 56: 960-967.
6. Rosso, A, et al. (2006). p53 Mediates the accelerated onset of senescence of endothelial progenitor cells in diabetes. J Biol Chem 281: 4339-4347.
7. Segal, M S, et al. (2006). Nitric oxide cytoskeletal-induced alterations reverse the endothelial progenitor cell migratory defect associated with diabetes. Diabetes 55: 102-109.
8. Gallagher, K A, et al. (2007). Diabetic impairments in NO-mediated endothelial progenitor cell mobilization and homing are reversed by hyperoxia and SDF-1 alpha. J Clin Invest 117: 1249-1259.
9. Busik, J V, et al. (2009). Diabetic retinopathy is associated with bone marrow neuropathy and a depressed peripheral clock. J Exp Med 206: 2897-2906.
10. Yamazaki, S, Iwama, A, Takayanagi, S, Eto, K, Ema, H, and Nakauchi, H (2009). TGF-beta as a candidate bone marrow niche signal to induce hematopoietic stem cell hibernation. Blood 113: 1250-1256.
11. Hatzfeld, J, et al. (1991). Release of early human hematopoietic progenitors from quiescence by antisense transforming growth factor beta 1 or Rb oligonucleotides. J Exp Med 174: 925-929.
12. Imbert, A M, Bagnis, C, Galindo, R, Chabannon, C, and Mannoni, P (1998). A neutralizing anti-TGF-beta1 antibody promotes proliferation of CD34+Thy-1+ peripheral blood progenitors and increases the number of transduced progenitors. Exp Hematol 26: 374-381.
13. Blank, U, et al. (2006). Smad7 promotes self-renewal of hematopoietic stem cells. Blood 108: 4246-4254.
14. Sitnicka, E, Ruscetti, F W, Priestley, G V, Wolf, N S, and Bartelmez, S H (1996). Transforming growth factor beta 1 directly and reversibly inhibits the initial cell divisions of long-term repopulating hematopoietic stem cells. Blood 88: 82-88.
15. Keller, J R, Mantel, C, Sing, G K, Ellingsworth, L R, Ruscetti, S K, and Ruscetti, F W (1988). Transforming growth factor beta 1 selectively regulates early murine hematopoietic progenitors and inhibits the growth of IL-3-dependent myeloid leukemia cell lines. J Exp Med 168: 737-750.
16. Bhatwadekar, A D, et al. (2010). Transient inhibition of transforming growth factor-beta1 in human diabetic CD34+ cells enhances vascular reparative functions. Diabetes 59: 2010-2019.
17. Kortlever, R M, Higgins, P J, and Bernards, R (2006). Plasminogen activator inhibitor-1 is a critical downstream target of p53 in the induction of replicative senescence. Nat Cell Biol 8: 877-884.
18. Glowinska, B, Urban, M, Koput, A, and Galar, M (2003). New atherosclerosis risk factors in obese, hypertensive and diabetic children and adolescents. Atherosclerosis 167: 275-286.
19. Binder, B R, et al. (2002). Plasminogen activator inhibitor 1: physiological and pathophysiological roles. News Physiol Sci 17: 56-61.
20. Cantley, L C (2002). The phosphoinositide 3-kinase pathway. Science 296: 1655-1657.
21. Kandel, E S, and Hay, N (1999). The regulation and activities of the multifunctional serine/threonine kinase Akt/PKB. Exp Cell Res 253: 210-229.
22. Mukai, Y, Wang, C Y, Rikitake, Y, and Liao, J K (2007). Phosphatidylinositol 3-kinase/protein kinase Akt negatively regulates plasminogen activator inhibitor type 1 expression in vascular endothelial cells. Am J Physiol Heart Circ Physiol 292: H1937-1942.
23. Basu, A, Menicucci, G, Maestas, J, Das, A, and McGuire, P (2009). Plasminogen activator inhibitor-1 (PAI-1) facilitates retinal angiogenesis in a model of oxygen-induced retinopathy. Invest Ophthalmol Vis Sci 50: 4974-4981.
24. Batard, P, et al. (2000). TGF-(beta)1 maintains hematopoietic immaturity by a reversible negative control of cell cycle and induces CD34 antigen upmodulation. J Cell Sci 113 (Pt 3): 383-390.
25. Van Obberghen-Schilling, E, Roche, N S, Flanders, K C, Sporn, M B, and Roberts, A B (1988). Transforming growth factor beta 1 positively regulates its own expression in normal and transformed cells. J Biol Chem 263: 7741-7746.
26. Czekay, R P, et al. (2011). PAI-1: An Integrator of Cell Signaling and Migration. Int J Cell Biol 2011: 562481.
27. Degryse, B, Neels, J G, Czekay, R P, Aertgeerts, K, Kamikubo, Y, and Loskutoff, D J (2004). The low density lipoprotein receptor-related protein is a motogenic receptor for plasminogen activator inhibitor-1. J Biol Chem 279: 22595-22604.
28. Demaison, C, et al. (2002). High-level transduction and gene expression in hematopoietic repopulating cells using a human immunodeficiency [correction of immunodeficiency] virus type 1-based lentiviral vector containing an internal spleen focus forming virus promoter. Hum Gene Ther 13: 803-813.
29. Aicher, A, et al. (2003). Essential role of endothelial nitric oxide synthase for mobilization of stem and progenitor cells. Nat Med 9: 1370-1376.
30. Li Calzi, S, et al. (2008). Carbon monoxide and nitric oxide mediate cytoskeletal reorganization in microvascular cells via vasodilator-stimulated phosphoprotein phosphorylation: evidence for blunted responsiveness in diabetes. Diabetes 57: 2488-2494.
31. Bowie, M B, McKnight, K D, Kent, D G, McCaffrey, L, Hoodless, P A, and Eaves, C J (2006). Hematopoietic stem cells proliferate until after birth and show a reversible phase-specific engraftment defect. J Clin Invest 116: 2808-2816.
32. Takatoku, M, et al. (2001). Avoidance of stimulation improves engraftment of cultured and retrovirally transduced hematopoietic cells in primates. J Clin Invest 108: 447-455.
33. Uchida, N, et al. (1997). The unexpected G0/G1 cell cycle status of mobilized hematopoietic stem cells from peripheral blood. Blood 89: 465-472.
34. Jetmore, A, et al. (2002). Homing efficiency, cell cycle kinetics, and survival of quiescent and cycling human CD34(+) cells transplanted into conditioned NOD/SCID recipients. Blood 99: 1585-1593.
35. Loomans, C J, et al. (2004). Endothelial progenitor cell dysfunction: a novel concept in the pathogenesis of vascular complications of type 1 diabetes. Diabetes 53: 195-199.
36. Thum, T, et al. (2007). Endothelial nitric oxide synthase uncoupling impairs endothelial progenitor cell mobilization and function in diabetes. Diabetes 56: 666-674.
37. Tepper, O M, et al. (2002). Human endothelial progenitor cells from type II diabetics exhibit impaired proliferation, adhesion, and incorporation into vascular structures. Circulation 106: 2781-2786.
38. Aicher, A, et al. (2003). Essential role of endothelial nitric oxide synthase for mobilization of stem and progenitor cells. Nat Med 9: 1370-1376.
39. Blazar, B R, Hirsch, R, Gress, R E, Carroll, S F, and Vallera, D A (1991). In vivo administration of anti-CD3 monoclonal antibodies or immunotoxins in murine recipients of allogeneic T cell-depleted marrow for the promotion of engraftment. J Immunol 147: 1492-1503.
40. Taswell, C (1981). Limiting dilution assays for the determination of immunocompetent cell frequencies. I. Data analysis. J Immunol 126: 1614-1619.
Aicher A., Heeschen C., Mildner-Rihm C., Urbich C., Ihling C., Technau-Ihling K., Zeiher A. M., and Dimmeler S. 2003. Essential role of endothelial nitric oxide synthase for mobilization of stem and progenitor cells.
Asahara, T., T. Murohara, A. Sullivan, M. Silver, R. Zee, and T. Li. 1997. Isolation of putative progenitor endothelial cells for angiogenesis. Science 275:964-967.
Asahara, T., T. Murohara, A. Sullivan, M. Silver, R. van der Zee, T. Li, B. Witzenbichler, G. Schatteman, and J. M. Isner. 1997. Isolation of Putative Progenitor Endothelial Cells for Angiogenesis. Science 275:964-966.
Basu, A., G. Menicucci, J. Maestas, A. Das, and P. McGuire. 2009. Plasminogen Activator Inhibitor-1 (PAI-1) Facilitates Retinal Angiogenesis in a Model of Oxygen-Induced Retinopathy. Invest. Ophthalmol. Vis. Sci. 50:4974-4981.
Basu, S., and H. E. Broxmeyer. 2005. Transforming growth factor-{beta} 1 modulates responses of CD34+ cord blood cells to stromal cell-derived factor-1/CXCL12. Blood 106:485-493.
Bhatwadekar, A. D., E. P. Guerin, Y. P. R. Jarajapu, S. Caballero, C. Sheridan, D. Kent, L. Kennedy, M. C. Lansang, F. W. Ruscetti, C. J. Pepine, P. J. Higgins, S. H. Bartelmez, and M. B. Grant. 2010. Transient Inhibition of Transforming Growth Factor-β1 in Human Diabetic CD 34+ Cells Enhances Vascular Reparative Functions. Diabetes
Binder, B. R., G. Christ, F. Gruber, N. Grubic, P. Hufnagl, M. Krebs, J. Mihaly, and G. W. Prager. 2002. Plasminogen activator inhibitor 1: physiological and pathophysiological roles. News Physiol. Sci. 17:56-61.
Busik, J. V., M. Tikhonenko, A. Bhatwadekar, M. Opreanu, N. Yakubova, S. Caballero, D. Player, T. Nakagawa, A. Afzal, J. Kielczewski, A. Sochacki, S. Hasty, S. L. Calzi, S. Kim, S. K. Duclas, M. S. Segal, D. L. Guberski, W. J. Esselman, M. E. Boulton, and M. B. Grant. 2009. Diabetic retinopathy is associated with bone marrow neuropathy and a depressed peripheral clock. J. Exp. Med. 206:2897-2906.
Caballero, S., N. Sengupta, A. Afzal, K. Chang, S. L. Calzi, D. Guberski, T. Kern, and G. B. Maria. 2007. Ischemic Vascular Damage Can Be Repaired by Healthy, but Not Diabetic, Endothelial Progenitor Cells. 56:
Cantley, L. C. 2002. The phosphoinositide 3-kinase pathway. Science 296:1655-1657.
Chen, Y. H., S. J. Lin, F. Y. Lin, T. C. Wu, C. R. Tsao, P. H. Huang, P. L. Liu, Y. L. Chen, and J. W. Chen. 2007. High Glucose Impairs Early and Late Endothelial Progenitor Cells by Modifying Nitric Oxide-Related but Not Oxidative Stress-Mediated Mechanisms. Diabetes 56:1559-1568.
Das, A., P. McGuire, C. Eriqat, R. Ober, E. DeJuan Jr, G. Williams, A. McLamore, J. Biswas, and D. Johnson. 1999. Human diabetic neovascular membranes contain high levels of urokinase and metalloproteinase enzymes. Invest. Ophthalmol. Vis. Sci. 40:809-813.
Fadini, G. P., S. V. de Kreutzenberg, A. Coracina, I. Baesso, C. Agostini, A. Tiengo, and A. Avogaro. 2006. Circulating CD34+ cells, metabolic syndrome, and cardiovascular risk. Eur. Heart J. 27:2247-2255.
Grant, M. B., E. A. Ellis, S. Caballero, and R. N. Mames. 1996. Plasminogen activator inhibitor-1 overexpression in nonproliferative diabetic retinopathy. Exp. Eye Res. 63:233-244.
Grant, M. B., P. E. Spoerri, D. W. Player, D. M. Bush, E. A. Ellis, S. Caballero, and W. G. Robison. 2000. Plasminogen activator inhibitor (PAI)-1 overexpression in retinal microvessels of PAI-1 transgenic mice. Invest. Ophthalmol. Vis. Sci. 41:2296-2302.
McGuire, P. G., T. R. Jones, N. Talarico, E. Warren, and A. Das. 2003. The Urokinase/Urokinase Receptor System in Retinal Neovascularization: Inhibition by A6 Suggests a New Therapeutic Target. Invest. Ophthalmol. Vis. Sci. 44:2736-2742.
Mukai, Y., C. Wang, Y. Rikitake, and J. K. Liao. 2007. Phosphatidylinositol 3-kinase/protein kinase Akt negatively regulates plasminogen activator inhibitor type 1 expression in vascular endothelial cells. Am. J. Physiol. Heart Circ. Physiol. 292:H1937-1942.
Nagi, D. K., L. J. McCormack, V. Mohamed-Ali, J. S. Yudkin, W. C. Knowler, and P. J. Grant. 1997. Diabetic retinopathy, promoter (4G/5G) polymorphism of PAI-1 gene, and PAI-1 activity in Pima Indians with type 2 diabetes. Diabetes Care 20:1304-1309.
Pandolfi, A., D. Cetrullo, R. Polishuck, M. M. Alberta, A. Calafiore, G. Pellegrini, E. Vitacolonna, F. Capani, and A. Consoli. 2001. Plasminogen Activator Inhibitor Type 1 Is Increased in the Arterial Wall of Type II Diabetic Subjects. Arterioscler. Thromb. Vasc. Biol. 21:1378-1382.
Schafer, K., M. R. Schroeter, C. Dellas, M. Puls, M. Nitsche, E. Weiss, G. Hasenfuss, and S. V. Konstantinides. 2006. Plasminogen activator inhibitor-1 from bone marrow-derived cells suppresses neointimal formation after vascular injury in mice. Arterioscler. Thromb. Vasc. Biol. 26:1254-1259.
Schmittgen, T. D., and K. J. Livak. 2008. Analyzing real-time PCR data by the comparative C_{T} method.
Segal, M. S., R. Shah, A. Afzal, C. M. Perrault, K. Chang, A. Schuler, E. Beem, L. C. Shaw, S. L. Calzi, J. K. Harrison, R. Tran-Son-Tay, and M. B. Grant. 2006. Nitric oxide cytoskeletal-induced alterations reverse the endothelial progenitor cell migratory defect associated with diabetes.
Stefansson, S., and D. A. Lawrence. 1996. The serpin PAI-1 inhibits cell migration by blocking integrin alpha V beta 3 binding to vitronectin. Nature 383:441-443.
Thum, T., D. Fraccarollo, M. Schultheiss, S. Froese, P. Galuppo, and J. D. Widder. 2007. Endothelial nitric oxide synthase uncoupling impairs endothelial progenitor cell mobilization and function in diabetes. Diabetes 56:666-674.
Thum, T., D. Fraccarollo, M. Schultheiss, S. Froese, P. Galuppo, J. D. Widder, D. Tsikas, G. Ertl, and J. Bauersachs. 2007. Endothelial nitric oxide synthase uncoupling impairs endothelial progenitor cell mobilization and function in diabetes.
Xiang, G., M. D. Schuster, T. Seki, A. A. Kocher, S. Eshghi, A. Boyle, and S. Itescu. 2004. Down-regulation of plasminogen activator inhibitor 1 expression promotes myocardial neovascularization by bone marrow progenitors. J. Exp. Med. 200:1657-1666.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

All references, including patent and non-patent literature, cited herein are incorporated by reference in their entirety.

1. A method of treating vascular lesions in a subject in need thereof, said method comprising:
procuring hematopoietic stem cells from said subject to obtain procured hematopoietic stem cells;
treating said procured hematopoietic stem cells, ex vivo, by blocking activity of PAI-1 in said stem cells to obtain treated hematopoietic stem cells;
administering said treated hematopoietic stem cells to said subject.

2. The method of claim 1, wherein said treating comprises subjecting said procured hematopoietic stem cells to an antisense nucleotide specific to an mRNA sequence encoding PAI-1.

3. The method of claim 1, wherein said treating comprises subjecting said procured hematopoietic stem cells to an antibody specific to PAI-1.

4. The method of claim 1, wherein said treating comprises subjecting said procured hematopoietic stem cells to siRNA.

5. The method of claim 1, wherein said treating comprises subjecting said procured hematopoietic stem cells to miRNA.

6. The method of claim 5, wherein said miRNA is miR-146a.

7. The method of claim 1, wherein said treating comprises subjecting said procured hematopoietic stem cells to TGF-β1 phosphorodiamidate morpholino oligomers (PMO).

8. The method of claim 1, wherein said treating comprises subjecting said procured hematopoietic stem cells to shRNA.

9. The method of claim 1, wherein said subject is diabetic.

10. The method of claim 1, wherein said procured hematopoietic stem cells are CD34+ cells.

11. The method of claim 1, wherein said vascular lesions are associated with diabetic retinopathy.

12. The method of claim 1, further comprising coadministration of a PAI-1 blocking agent.

13. The method of claim 1, wherein said vascular lesions are associated with Retinal Vein Occlusion.

14. The method of claim 1, wherein said vascular lesions are associated with choroidal neovascularization.

15. A method of diminishing diabetic retinopathy in a subject comprising administering hematopoietic stem cells treated with a PAI-1 blocking agent to said subject.

16. The method of claim 15, wherein said administering comprises parenterally injecting cells or by intraoptic injection.

17. A method of enhancing repair of vessel lesion in a subject comprising administering hematopoietic stem cells treated with a PAI-1 blocking agent to said subject.

18. The method of claim 17, wherein said administering comprises parenterally injecting cells.

19. The method of claim 17, wherein said hematopoietic cells are autologous or allogeneic in origin.

20. The method of claim 1 wherein administering occurs in response to a stroke in said subject.

21-37. (canceled)
